(19) United States
(12) Patent Application Publication
(10) Pub. No.: US 2026/0345034 A1
(43) Pub. Date: Nov. 5, 2026

(54) HEARING DIAGNOSTIC SYSTEM

(71) Applicant: Analog Devices, Inc., Wilmington, MA (US)

(72) Inventors: Jean-Marc LUNEAU, Woluwe Saint Lambert (BE); Mikael Mortensen, Milpitas, CA (US)

(21) Appl. No.: 19/738,526

(22) Filed: Jul. 12, 2026

Related U.S. Application Data
(63) Continuation-in-part of application No. 18/529,822, filed on Dec. 5, 2023.
Application No. 18/529,822 is a continuation of application No. 16/862,864, filed on Apr. 30, 2020, now Pat. No. 11,864,886.
(60) Provisional application No. 62/840,645, filed on Apr. 30, 2019.

(51) Int. Cl. A61B 5/12; A61B 5/00; H04R 1/10

(52) U.S. Cl. CPC A61B 5/125; A61B 5/6803; A61B 5/7217; H04R 1/1083; H04R 1/1091; A61B 2560/0223; H04R 2460/01

(57) ABSTRACT

Technologies are provided for hearing testing and post-testing use that incorporates contextual information associated with a subject undergoing a hearing test and/or an environment in which the hearing test is performed or the subject uses a hearing device configured according to results of the hearing test. The hearing test may be implemented within headphones that, after the test, may serve as a hearing device for the subject. The contextual information may include, for example, physiological data of the subject, acoustic characteristics of the environment, and/or anatomical characteristics of the subject. Implementation of such a hearing test in a headphone limits the amount of calibration for accurate measurement when performing the hearing test and provides a comprehensive assessment of hearing ability of the subject under different hearing contexts. The headphone configured according to the hearing test accounts for various hearing contexts and provides robust performance in post-testing use.

### RELATED APPLICATIONS

This application is a continuation-in-part of U.S. Patent Application No. 18/529,822, filed Dec. 5, 2023, which application is a continuation of U.S. Patent Application No. 16/862,864 (now U.S. Patent No. 11,864,886), filed Apr. 30, 2020, which claims priority to U.S. Provisional Patent Application No. 62/840,645, entitled “HEARING DIAGNOSTIC SYSTEM,” and filed Apr. 30, 2019, the disclosures of which applications are incorporated by reference herein in their entireties.

### FIELD OF THE DISCLOSURE

This disclosure relates in general to the field of hearing diagnostics, and more particularly, though not exclusively, to systems and methods for self-calibration of audio devices that can be utilized for hearing diagnostics.

### BACKGROUND

Legacy hearing testing has involved visiting a special testing facility where hearing could be tested in a controlled environment to ensure accurate results of the testing. Having to visit a special testing facility can be time consuming and can limit a tested population due to the availability of the special testing facilities and the amount of tests that can be performed in a time period by the special testing facility.

### SUMMARY OF THE DISCLOSURE

Herein disclosed are systems and methods that, individually or in combination, can be implemented within headphones to permit or otherwise facilitate hearing testing and use of hearing devices post-testing. Implementation of the system in the headphone may include implementation of tone generation circuitry and sound pressure level measurement circuitry being implemented in the headphone limiting the amount of calibration for accurate measurement when performing a hearing test. In addition, or in some cases, implementation of the systems in the headphone can include implementation of one or multiple modules that can determine information representing or otherwise defining a context of the headphone. Such information may be generated during a hearing test and may be utilized to augment results of the hearing test. Such information also may be generated during post-testing use of the headphone, to adjust performance of the headphone based on one or more types of contexts.

### BRIEF DESCRIPTION OF THE DRAWINGS

The present disclosure is best understood from the following detailed description when read with the accompanying figures. It is emphasized that, in accordance with the standard practice in the industry, various features are not necessarily drawn to scale, and are used for illustration purposes only. Where a scale is shown, explicitly or implicitly, it provides only one illustrative example. In other embodiments, the dimensions of the various features may be arbitrarily increased or reduced for clarity of discussion.

FIG. 1 illustrates hearing profiles of average individuals based on age.

FIG. 2 illustrates example hearing profiles for the types of hearing loss.

FIG. 3 illustrates hearing loss that may be experienced by carpenter due to occupational noise exposure.

FIG. 4 illustrates a loudness compensation based on Fletcher-Munson Contours.

FIG. 5 illustrates an example system that can implement the approaches described throughout this disclosure, according to some embodiments.

FIG. 6 illustrates an example system that can implement the approaches described throughout this disclosure, according to some embodiments.

FIG. 7 illustrates an example system that can implement the approaches described throughout this disclosure, according to some embodiments.

FIG. 8 illustrates an example system that can implement the approaches described throughout this disclosure, according to some embodiments.

FIG. 9 illustrates an example system that can implement the approaches described throughout this disclosure, according to some embodiments.

FIG. 10 illustrates an example earbud, according to various embodiments.

FIG. 11 illustrates an example hearing test procedure, according to various embodiments.

FIG. 12 illustrates an example system that can implement the approaches described throughout this disclosure, according to some embodiments.

FIG. 13 illustrates an example system that can implement the approaches described throughout this disclosure, according to some embodiments.

FIG. 14 illustrates an example system that can implement the approaches described throughout this disclosure, according to some embodiments.

FIG. 15 is a flowchart of an example of a method for performing a hearing test in accordance with one or more aspects of this disclosure.

FIG. 16 is a flowchart of another example of a method for performing a hearing test in accordance with one or more aspects of this disclosure.

FIG. 17 is a flowchart of an example of a method for compensating for hearing loss in accordance with one or more aspects of this disclosure.

### DETAILED DESCRIPTION

Hearing and hearing loss are a growing issue in society. The advent of mobile devices and headphones is driving loudness related hearing loss towards younger and younger age groups. The growth in the population experiencing hearing loss creates a desire for more and wider availability of hearing testing to identify hearing loss in individuals.

To help facilitate better hearing among a wider population, it is beneficial to be able to easily diagnose hearing. Legacy hearing testing involves visiting a specialist and having a test performed in a controlled environment. This to ensure the accuracy of the measurement results. Having to visit a specialist for hearing testing can be onerous and time consuming.

Self-administered hearing tests using conventional headphones have shown to be feasible within an acceptable error margin as a test vehicle. In particular, assuming that the entire signal chain has been re-calibrated with the conventional headphones, the approach can be feasible.

Conventional headphones used were calibrated and the test software adjusted, to ensure that the output sound pressure level (SPL) in the self-administered test can match the fully calibrated standard measurement system administered by the specialist in the legacy hearing tests.

Ensuring that the full acoustic signal chain is calibrated is a significant challenge. Further challenges may include the frequency characteristics of the headphones themselves, and the environment in which the measurements are performed. These factors can matter if an accurate measurement is to be performed.

### Calibrated Signal Chain

For a closed system, it is possible to pre-calibrate the system, such that the SPL of the tones issued into the ear are guaranteed to be a given value. In particular, pre-calibration of a closed system can be utilized to provide for accurate hearing testing without the losses and/or inaccuracies that can occur in non-closed systems.

In a system where the source of test tones is, for example, a smart phone (also referred to as smartphone), and the transducer is a mobile headphone, it can be difficult to ensure that when a particular SPL value is assumed to be played back by the mobile headphone, that the particular SPL value is actually present. For example, conversions of the test tones between the smartphone and the mobile headphone may cause changes in an intended SPL value that can cause an actual SPL value of the test tone that is presented by the mobile headphone to be different than the intended SPL value of the test tone.

Introducing a feedback signal path, from an ear, that allows the system to continuously monitor the SPL of the issued tone, can ensure that a desired SPL to be played back by the headphone is actually present. Allowing the system to adjust to gain to the desired SPL can result in a more correct measurement.

The feedback signal path can include a microphone placed in the ear as part of the transducer. For example, the microphone can be placed within a headphone, such as within a silicone plug of an earbud and/or within the headphone itself, where being placed in the silicone plug can provide little environmental noise. This microphone, if pre-calibrated, can provide a measurement of the SPL in the test subject’s ear. Further, circuitry within the headphone can determine an SPL measured by the microphone within the headphone and compensate for a difference between the measured SPL and the SPL intended to be emitted by the headphone. In some embodiments, tone generator circuitry and SPL measurement circuitry that may be implemented within the headphone may eliminate inaccuracies that can be caused by transmitting test tones between a smartphone and headphones.

### Frequency Characteristic of the Headphones

Normally, part of the signal chain calibration can include the headphones, and hence the frequency characteristic of the headphones and their influence on the measurement result and/or apply linearization filters can be determined. However, as headphones are changed out and/or characteristics of headphones may change due to use, calibration may need to be performed again to maintain accurate hearing measurements.

With no change, the frequency characteristic of the headphone can affect the measurement result. Having a calibrated microphone in the signal path allows for a dynamic adjustment of the output amplitude to compensate for the headphone.

### Measurement Environment

The environment in which the user is when the measurement is performed can impact the result. This is why users are placed in a sound proof booth at an audiologist during legacy hearing testing.

For a consumer-based hearing test headset there can be two methods available for controlling the environment. The first is passive attenuation by blocking environmental sounds from the user’s ears. The second is to apply Active Noise Cancellation (ANC) to remove the undesired environmental noise from the user’s ears.

In some embodiments, ANC is implemented, and the in-ear microphone can be part of a feedback ANC system. In particular, the microphone located within the headphone may detect environmental noise and provide the data related to the detected environmental noise to the system. The system may then apply noise cancellation based on the data related to the detected environmental noise to cancel out the environmental noise.

### Microphone Type

It is possible to use any microphone for the system, but it may be beneficial to utilize microelectro-mechanical systems (MEMS) microphones. MEMS microphones may have a nice flow frequency characteristic and good aging effects. For example, the MEMS microphones may have a flat frequency response curve in the range of frequencies that may be utilized for hearing testing.

Utilizing a good microphone (such as a MEMS microphone) can make the rest of the system cheaper to manufacture since the need for per unit calibration can be a lot lower. With a MEMS microphone it is feasible to make a self-calibrating system.

A challenge presented by a MEMS microphone is the maximum SPL it can withstand. Here a multi-element MEMS microphone akin to high SPL MEMS microphone could be beneficial.

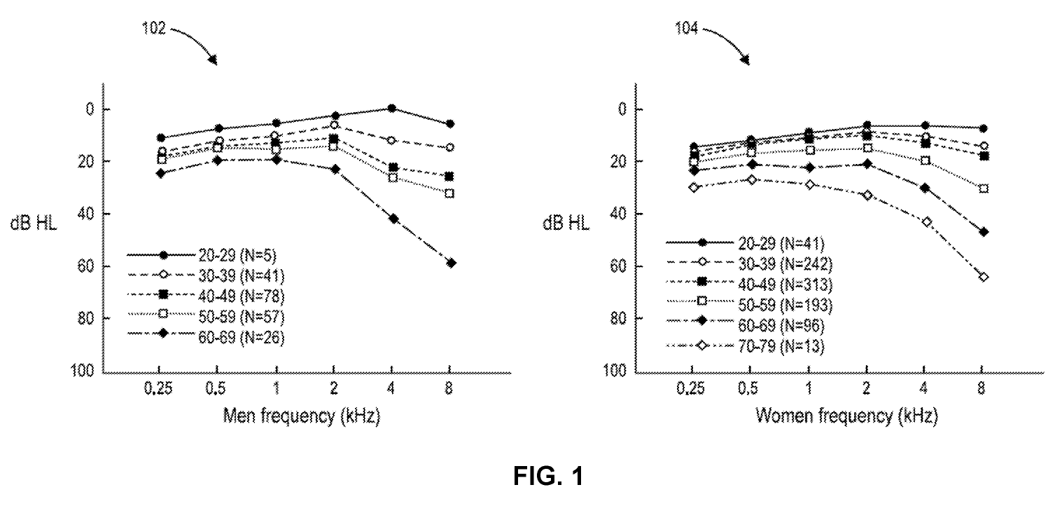

When a hearing profile has been attained via measurement it is possible to compare the hearing profile to an average hearing loss profile based on sex and age. Any large deviation from this average profile can be indicative of a hearing condition. Depending on the deviation, a preliminary diagnosis can be provided. FIG. 1 illustrates hearing profiles of average individuals based on age. In particular, chart 102 illustrates hearing profiles for males of different ages. Chart 104 illustrates hearing profiles for females of different ages.

One type of hearing loss is conductive hearing loss. Conductive hearing loss results from any condition in the outer or middle ear that interferes with sound passing to the inner ear. Some conditions that can cause conductive hearing loss include excessive wax in the auditory canal, a ruptured eardrum, and work-related conductive hearing loss (such as head injuries, penetration of the eardrum by a sharp object, or any event that ruptures the eardrum or breaks the ossicular chain formed by small bones in the middle ear). Conductive hearing loss can include relatively uniform reduced hearing across all frequencies in hearing tests, with no reduction when sounds are transmitted through bone conduction. Conductive hearing loss may be reversible via medical or surgical treatment.

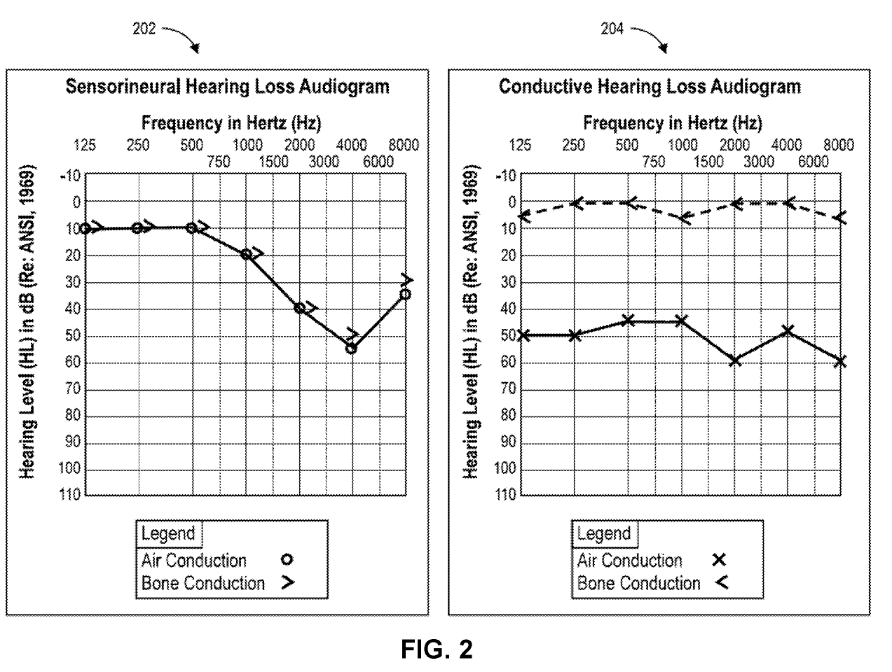

Another type of hearing loss is sensorineural hearing loss. Sensorineural hearing loss can be a permanent condition and can be associated with irreversible damage to the inner ear. Sensorineural hearing loss can be caused by the normal aging process and/or excessive noise exposure. FIG. 2 illustrates example hearing profiles for the types of hearing loss. In particular, chart 202 illustrates example hearing profiles for sensorineural hearing loss. Chart 204 illustrates example hearing profiles for conductive hearing loss.

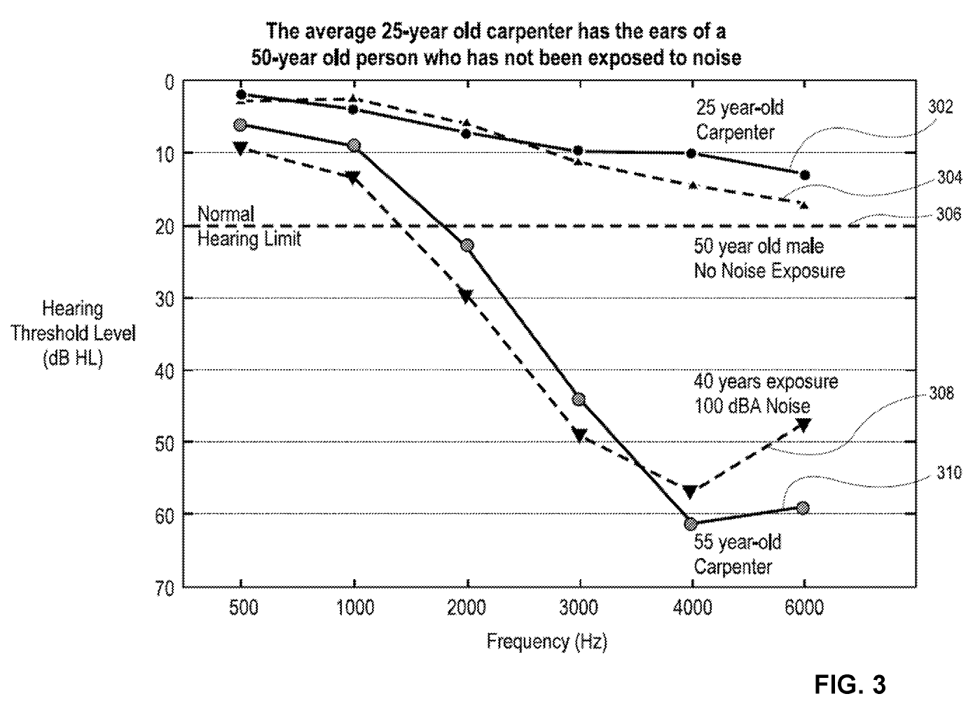

Sensorineural hearing loss can be caused by occupational noise exposure in some instances. FIG. 3 illustrates hearing loss that may be experienced by carpenters due to occupational noise exposure. Line 302 illustrates a hearing profile of a 25-year old carpenter. Line 304 illustrates a hearing profile of a 50-year old male with noise exposure. Line 306 illustrates a threshold of what is considered a normal hearing limit. Line 308 illustrates a hearing profile of an individual who has experienced 40 years of exposure to 100 A-weighted decibels (dBA) of noise. Line 310 illustrates a hearing profile of 55-year old carpenter.

For hearing profiles, any loss can be a temporary effect, which can also be known as temporary threshold shifts.

Hence a single measurement in isolation may not be enough. In a clinical environment, besides an audiogram measurement, there will also be an interview of recent high noise exposure and a visual inspection of the ear to rule out hearing loss based on visually detectable factors, such as ear wax and disease.

For self-administered hearing tests, it may be beneficial to ensure that not just a single measurement is performed, but also that any large deviation from the norm results in a recommendation to visit a doctor.

### Example of Self-assessment for Medical Diagnostics

A further reason to perform continuous measurements of hearing state can be exemplified by cancer treatments. Cancer treatments can lead to hearing loss, where the reason for hearing loss is partially known (platinum-based ototoxicity, for example) and partially unknown.

As treatment-based hearing loss may not occur momentarily, having a self-administered hearing test to keep track of any potential changes in hearing for a cancer patient under treatment can be used to adjust the cancer treatment to prevent further damage.

### Compensation of Hearing Loss

When an audiogram has been measured by the user via self-assessment it is possible to perform various compensation techniques. For example, the system within the headphone may apply compensation for sounds to be emitted from the headphones based on an audiogram of an individual that may be produced based on hearing testing of the individual. The audiogram utilized for compensation may have been produced from hearing testing performed by the system.

### Equalizer

A first technique of performing compensation is to apply an equalization technique. Here the desired audio signal may be attenuated with the inverted audiogram to create a linear frequency characteristic. An additional technique is to add a loudness filter on top based on the measured SPL/volume setting. This may make the sound not only more natural but it may also drive a more natural/lower hearing level to potentially prevent further loss of hearing.

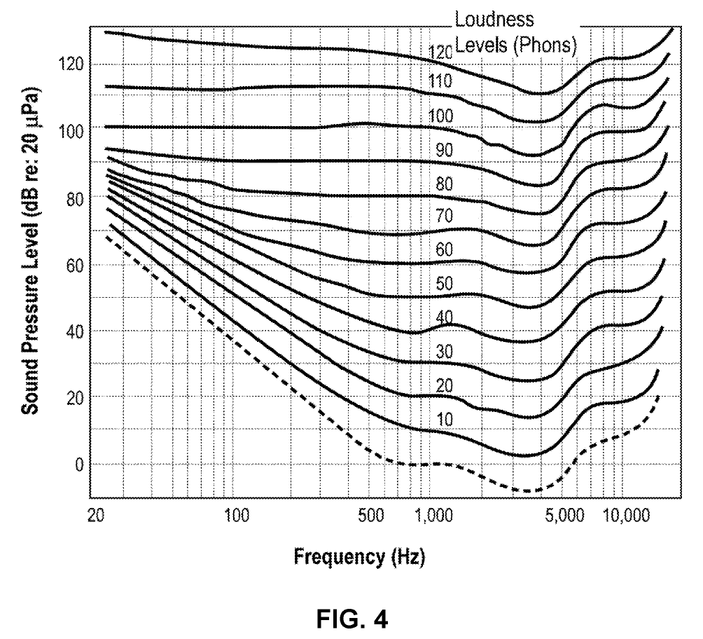

For dramatic hearing loss compensation, a gradual change may be introduced. This as the brain of the user is already trying to compensate for the loss of hearing. Hence a sudden correction can be disconcerting and even painful for the user. FIG. 4 illustrates a loudness compensation based on Fletcher-Munson Contours. The contours show the SPL at each frequency to produce the same loudness in an average listener. The actual threshold may vary by as much as 10 decibels in either direction.

### Compression and Expansion

To help with some aspects of hearing loss, equalization may not be enough. Instead, compression of the spectrum can be used. This is a similar thing to the compression seen in frequency modulation (FM)/amplitude modulation (AM) and telephony to adjust for the limited bandwidth of the transmission system.

Compression can be used to conceptually move parts of the sound spectrum that a user cannot hear down into parts of the spectrum that the user can hear in a way that makes it sound natural, to the extent possible.

Expansion can be a method for removing low level sounds from the user of the device. Compensation can involve some level of amplification of the incoming sound. Said amplification also includes low levels of noise/sound, which, if the user is in a quiet environment, can become uncomfortable/strange. Expansion can be added to prevent what can be called “super hearing.”

### Measuring an Audiogram BIAS and BIAS Prevention

For the system a feature is how the audiogram is measured. Assuming the system has been designed as a self-calibrating system with environmental protections included, it can be used to perform self-assessment of hearing.

### Standard BIASed Approach

The standard approach for measuring hearing is to subject the user to a sequence of log spaced (for example, mel spaced or cochlear response) sine tones at a moderate sound SPL, followed by a decrease in SPL based on user feedback.

For example, the user is exposed to a 1 kilohertz (kHz) tone at 84 decibels (dB) SPL, if the user acknowledges the tone is heard, the SPL is decreased, and the test repeated until the user cannot hear the tone.

A challenge with the standard approach is that it can introduce an inherent BIAS. Depending on the methodology used, the user’s brain can predict the tone issue time and create a phantom tone for the user to hear, even when no tone is actually heard. This BIAS results in a distorted audiogram.

For self-assessment often a direct volume dial and/or volume control is available to the user. This can also lead to a BIAS, as the user knows a tone is playing. This can again cause the brain to create a phantom tone that can distort the audiogram.

For fast testing, the number of bands used can be quite low. With a low number of test bands, the resulting audiogram, even though good enough for rudimentary diagnostics, can be too crude for proper compensation.

### Unbiased Testing Approaches

To remove bias from the hearing test, predictability can be removed, basically preventing the brain of the user to pattern out the test.

Examples of this is to randomize parts of the test parameters such that the user is not exposed to the same frequency and/or SPL at any given time. For a system like this, it is what the user cannot hear can be maintained (such as in memory), such that the user does not often hear “nothing.” Hearing nothing when a sound is expected can be disconcerting and can cause the user to just press the feedback button.

To minimize search patterns one could use, for example, binary search trees to fill in the audiogram.

Other approaches include using band passed sounds like chirps (which can be referred to as band limited testing). In these approaches, the user can be partially trained in recognizing chirps and what a distorted chirp sounds like. An advantage of using chirps is that it dramatically reduces the audiogram test time.

More advanced tests like speech testing and/or environmental test/experiences can be applied to further remove bias from the testing approach.

### Smartphone Based Testing and Compensation

Adding a smartphone into the system can be a seemingly easy way to add a lot of advanced features, like the test generation and compensation. A challenge for smartphones can be the acoustic path from the phone itself to the headset.

The signal path from the smartphone often includes a wireless/Bluetooth (BT) path in which audio compression can be performed. Audio compression can involve applying a psycho-acoustic filter to the audio being compressed, which distorts the measurements.

The audio from the microphone could potentially be used to fight the distortion of the audio compression, with the caveat that the microphone audio itself can be exposed to audio compression.

Further complexity comes from the fact that most cellphone audio paths themselves may include some sort of equalization to create a specific “sound” that allows a given brand to be easily recognized by the user.

A smartphone system could be constructed so that it does not face the above challenges, but for the legacy smartphones this will not be the case.

Instead, the test tones, SPL measurements, and compensation may be created in the headset itself. This can be done, for example, using a specially designed integrated circuit (such as a processor), or a combination of local signal processing and measurement in the headset.

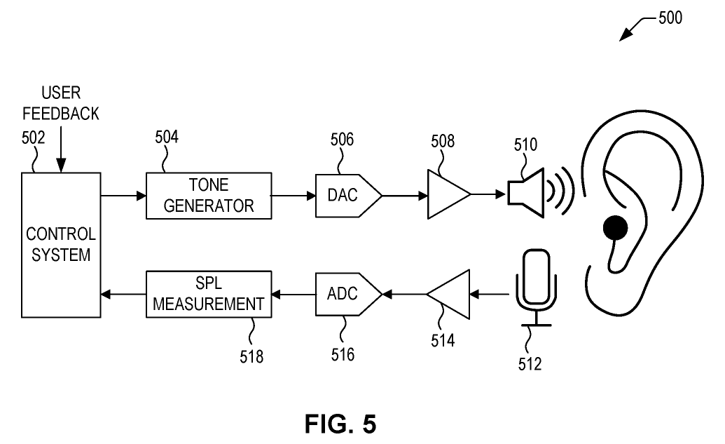

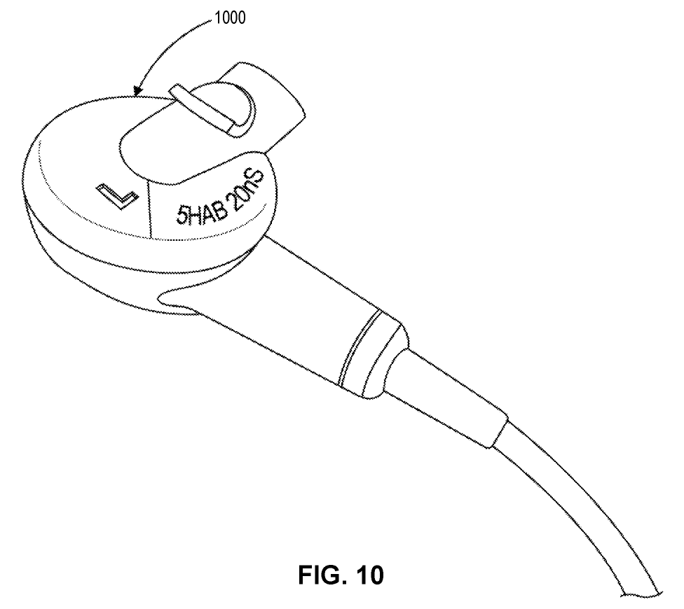

FIG. 5 illustrates an example system 500 that can implement the approaches described throughout this disclosure, according to some embodiments. For example, the system 500 may implement the hearing testing approaches, the unbiased testing approaches, and/or the compensation approaches described throughout this disclosure. The system 500 can be implemented in headphones, including an earphone or an earbud of the headphones, such as the earbud 1000 (FIG. 10). In some embodiments, the system 500, or some portion thereof, may be implemented in a processor located within the headphones. In some embodiments, a single earphone or earbud of the headphones may include the system 500, whereas each of the earphones or earbuds of the headphone may include the system 500 in other embodiments.

The system 500 can include a control system 502. The control system 502 can control one or more elements of the system 500. Further, the control system 502 can receive user feedback. The user feedback may indicate that the user heard a tone, the user did not hear a tone, or some combination thereof. The control system 502 may be coupled to a remote device (such as a smart phone) and may communicate with the remote device. The control system 502 may initiate a hearing test in response to a trigger from the remote device, or may initiate a hearing test in response to a user interaction with the headphone, such as the user pressing a button on the headphone.

The system 500 can further include a tone generator 504. The tone generator 504 can generate tones for determining calibration of the system 500. Further, the tone generator 504 can generate tones for hearing testing of the user. The tone generator 504 can generate the tones as digital signals. The tone generator 504 may generate the tones for calibration of the system 500 and/or for the hearing testing of the user in response to signals received from the control system 502.

The tone generator 504 can be coupled to the control system 502 and the control system 502 can cause the tone generator 504 to generate the tones. For example, the control system 502 can output a signal to the tone generator 504 to cause the tone generator 504 to generate a tone that corresponds to the signal. The amplitude and/or frequency of the tone generated by the tone generator 504 may be based on a value of the signal output by the control system 502, an amplitude of the signal output by the control system 502, a frequency of the signal output by the control system 502, a timing offset of the signal output by the control system 502, a voltage offset of the signal output by the control system 502, or some combination thereof. In particular, as the value, amplitude, frequency, timing offset, and/or voltage offset of the signal output by the control system 502 is changed, the tone generator 504 may change the amplitude, frequency, and/or timing offset of the tone. The control system 502 can change the value, amplitude, frequency, timing offset, and/or voltage offset of the signal output by the control system 502 by predefined intervals, according to a predefined algorithm, or randomly change the characteristics.

The system 500 can further include a digital-to-analog converter (DAC) 506. The DAC 506 can be coupled to the tone generator 504 and can receive the digital signals corresponding to the tones from the tone generator 504. The DAC 506 can convert the digital signals received from the tone generator 504 to analog signals. In some embodiments, the tone generator 504 can generate analog signals and the DAC 506 can be omitted.

The system 500 can further include a buffer 508. The buffer 508 may be coupled to the DAC 506 and may receive the analog signals corresponding to the tones from the DAC 506. The buffer 508 may buffer the analog signals and, in some embodiments, can alter an amplitude of the analog signals.

The system 500 can further include a speaker 510. In some embodiments, the speaker 510 may be a speaker of the headphones utilized for producing sounds for a user. The speaker 510 may be coupled to the buffer 508 and may receive the analog signals corresponding to the tones generated by the tone generator 504 from the buffer 508. The speaker 510 may emit sound based on the received analog signals. For example, the speaker 510 may emit the sounds corresponding to the tones generated by the tone generator 504 based on the received analog signals.

SPLs of the sounds emitted by the speaker 510 may be dependent on the amplitude and/or frequency of the tones generated by the tone generator 504. Further, as previously described, the amplitude and/or the frequency of the tones are dependent on the signal output by the control system 502. Accordingly, the SPLs of the sounds emitted by the speaker 510 are dependent on the signal output by the control system 502. The control system 502 can be unaware of the SPLs of the sounds to be output by the speaker 510 in response to the signals output by the control system 502. For example, the system 500 may not implement a calibration procedure, which can be required for the control system 502 to be aware of the SPLs of the sounds to be output by the speaker 510 in response to the signals output by the control system 502. In implementing a procedure for hearing testing of a user, the control system 502 may output a signal to the tone generator 504 in accordance with a predefined characteristic scheme or randomly selected without consideration of the SPL of the sound to be output by the speaker 510 corresponding to the signal. The control system 502 may continue to output signals without knowledge of the SPLs of the sounds to be output by the speaker 510 corresponding to the signals.

The system 500 can further include a microphone 512. In some embodiments, the microphone 512 may be a MEMS microphone. The microphone 512 may receive the sound emitted by the speaker 510. For example, the microphone 512 may receive the tones emitted by the speaker 510. The microphone 512 may convert the sounds to an analog, electrical signal.

The system 500 can further include a buffer 514. The buffer 514 may be coupled to the microphone 512 and may receive the analog signals from the microphone 512. The buffer 514 may buffer the analog signals and, in some embodiments, can alter an amplitude of the analog signals.

The system 500 can further include an analog-to-digital converter (ADC) 516. The ADC 516 may receive the analog signals from buffer 514 and convert the analog signals to digital signals.

The system 500 can further include an SPL measurement element 518. The SPL measurement element 518 may be coupled to the ADC 516 and may receive the analog signals from the ADC 516. The SPL measurement element 518 may determine the SPL of the sounds received by the microphone 512 based on the analog signals. For example, the SPL measurement element 518 may determine the SPL of the tones received by the microphone 512 based on the analog signals.

The control system 502 may be coupled to the SPL measurement element 518 and may receive indications of the SPL determined by the SPL measurement element 518 corresponding to each of the tones. Further, the control system 502 may receive user feedback from the user indicating whether the user heard the tone emitted by the speaker 510. For example, the speaker 510 may emit a tone corresponding to a signal output by the control system 502. The microphone 512 may detect the tone and the SPL measurement element 518 can determine an SPL of the tone. The control system 502 may receive an indication whether the user heard the tone and the SPL of the tone determined by the SPL measurement element 518. The control system 502 may store a data element indicating the determined SPL of the tone and whether the user heard the sound. The control system 502 may continue to output different signals, receive indications of the SPLs of the tones detected by the microphone 512 corresponding to each of the signals output by the control system 502, receive indications whether each of the tones corresponding to the signals were heard by the user, and store data elements for each of the signals indicating the determined SPL of the tone and whether the user heard the tone until the control system 502 determines that the hearing test has been completed. The control system 502 may determine that the hearing test has been completed based on all the signals for a hearing test being output by the control system 502 and/or based on a determination by the control system 502 that an SPL at which the user stops hearing the tones has been determined within a predetermined precision (such as determining that SPL at which user stops hearing the tones can be identified within 5 dBs).

The elements of the system 500 may be co-located within or otherwise integrated into an earphone or an earbud (such as the earbud 1000, FIG. 10). For example, the speaker 510 and the microphone 512 may be co-located within or otherwise integrated into the earphone or the earbud. The speaker 510 and the microphone 512 may be directed in the same direction. For example, the speaker 510 and the microphone 512 both may be directed toward an ear of a user of the earphone or earbud when the earphone or earbud is worn by the user. Further, the control system 502, the tone generator 504, the DAC 506, the buffer 508, the buffer 514, the ADC 516, and/or the SPL measurement element 518 may be implemented within one or more integrated circuits (ICs) (such as processors) that are located within the earphone or the earbud.

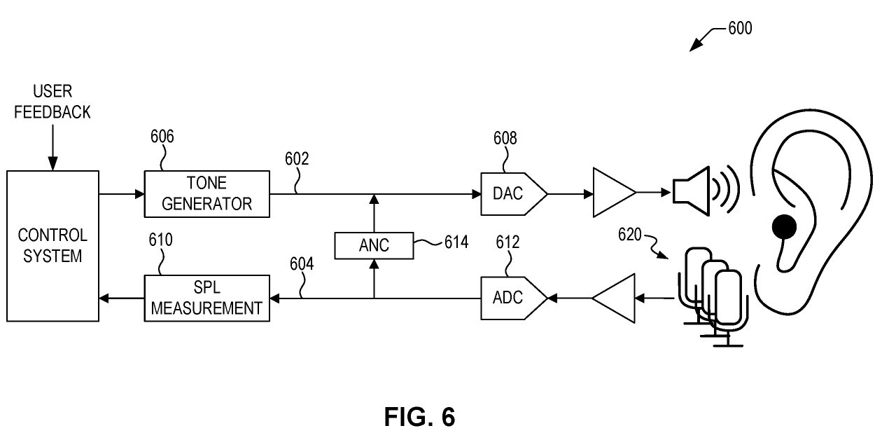

FIG. 6 illustrates an example system 600 that can implement the approaches described throughout this disclosure, according to some embodiments. For example, the system 600 may implement the hearing testing approaches, the unbiased testing approaches, and/or the compensation approaches described throughout this disclosure. The system 600 can be implemented in headphones, including an earphone or an earbud of the headphones, such as the earbud 1000 (FIG. 10). In some embodiments, the system 600, or some portion thereof, may be implemented in a processor located within the headphones. In some embodiments, a single earphone or earbud of the headphones may include the system 600, whereas each of the earphones or earbuds of the headphones may include the system 600 in other embodiments. The system 600 can include the same elements of the system 500 (FIG. 5).

The system 600 can include a first coupling element 602 (such as a wire, a trace, or other electrically-conductive elements) that couples a tone generator 606 and a DAC 608, where the tone generator 606 and the DAC 608 have the same features as the tone generator 504 (FIG. 5) and the DAC 506 (FIG. 5), respectively. The system 600 can further include a second coupling element 604 that couples an SPL measurement element 610 and an ADC 612, where the SPL measurement element 610 and the ADC 612 have the same features as the SPL measurement element 518 (FIG. 5) and the ADC 516 (FIG. 5), respectively. The system 600 further includes an ANC element 614. The ANC element 614 is coupled between the first coupling element 602 and the second coupling element 604. The ANC element 614 may detect the signal on the second coupling element 604 and apply noise cancelling to the signal on the first coupling element 602. The ANC element 614 may be co-located within or otherwise integrated into the earphone or the earbud with the other elements. The ANC element 614 uses, in some cases, a feedback microphone that is part of one or more microphones 620 included in the system 600. The feedback microphone generates an analog signal that results in the signal on the second coupling element 604. In other cases, the ANC element 614 uses a feed-forward microphone that is part of the one or more microphones 620. The feed-forward microphone generates an analog signal that results in the signal on the second coupling element 604. In still other cases, the ANC element 614 uses both a feedback microphone and a feed-forward microphone that are part of the one or more microphones 620. In some embodiments, the IC or ICs that implement other elements of the system 600 can implement the ANC element 614. For example, the IC or ICs may include a control element (such as the control system 502 (FIG. 5)), the tone generator 606, the first coupling element 602, the second coupling element 604, the DAC 608, the ADC 612, the SPL measurement element 610, and/or one or more buffers (such as the buffer 508 (FIG. 5) and the buffer 514 (FIG. 5)).

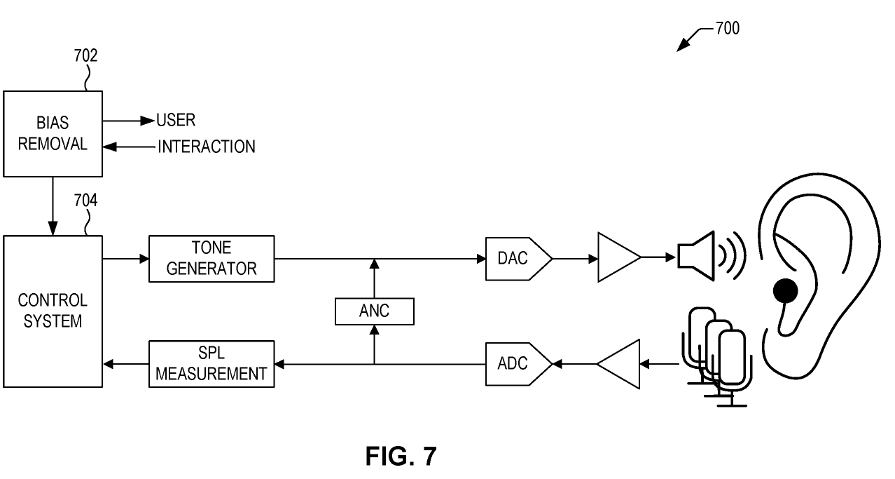

FIG. 7 illustrates an example system 700 that can implement the approaches described throughout this disclosure, according to some embodiments. For example, the system 700 may implement the hearing testing approaches, the unbiased testing approaches, and/or the compensation approaches described throughout this disclosure. The system 700 can be implemented in headphones, including an earphone or an earbud of the headphones, such as the earbud 1000 (FIG. 10). In some embodiments, the system 700, or some portion thereof, may be implemented in a processor located within the headphones. In some embodiments, a single earphone or earbud of the headphones may include the system 700, whereas each of the earphones or earbuds of the headphone may include the system 500 in other embodiments. The system 700 can include the same elements of the system 600 (FIG. 6).

The system 700 can further include a BIAS removal element 702. The BIAS removal element 702 can be coupled to a control system 704, where the control system 704 has the same features as the control system 502 (FIG. 5). The BIAS removal element 702 may implement a BIAS removal approach and/or the unbiased testing approach, as described throughout this disclosure. The BIAS removal element 702 may be co-located within or otherwise integrated into the earphone or the earbud with the other elements of the system 700. In some embodiments, the IC or ICs that implement other elements of the system 700 can implement the BIAS removal element 702.

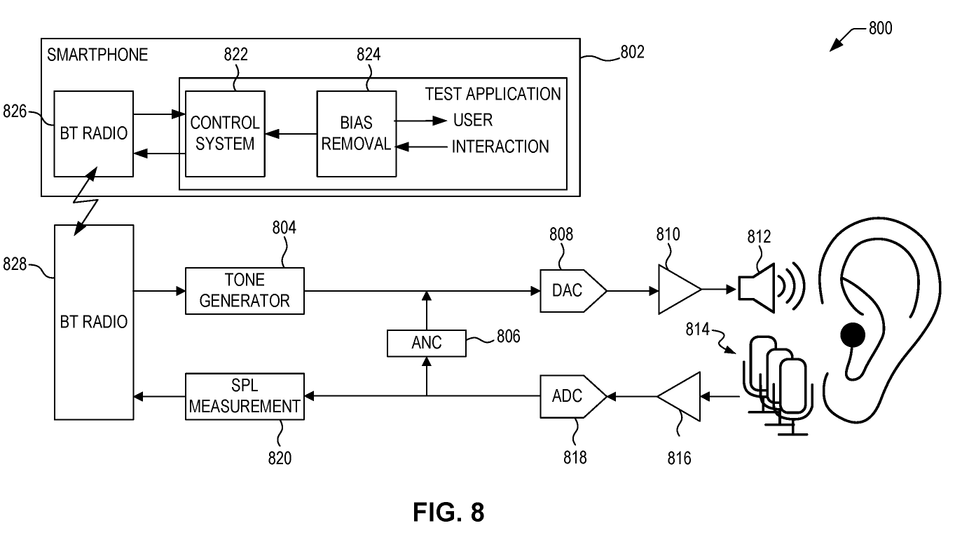

FIG. 8 illustrates an example system 800 that can implement the approaches described throughout this disclosure, according to some embodiments. For example, the system 800 may implement the hearing testing approaches, the unbiased testing approaches, and/or the compensation approaches described throughout this disclosure. The system 800, or some portion thereof, can be implemented in headphones, including an earphone or an earbud of the headphones, such as the earbud 1000 (FIG. 10). In some embodiments, the system 800, or some portion thereof, may be implemented in a processor located within the headphones. In some embodiments, a single earphone or earbud of the headphones may include the system 800, whereas each of the earphones or earbuds of the headphone may include the system 800 in other embodiments. The system 800 can include the elements of the system 700 (FIG. 7), although the elements of the system 800 may be separated between the earphone or earbud and a mobile device 802, such as a smartphone. For example, the system 800 can include a tone generator 804, an ANC element 806, a DAC 808, a buffer 810, a speaker 812, one or more microphones 814, a buffer 816, an ADC 818, and a SPL measurement element 820 co-located within or otherwise integrated into the earphone or earbud. Further, the system 800 can include a control system 822 and a BIAS removal element 824 co-located within or otherwise integrated into the mobile device 802. For example, the control system 822 and the BIAS removal element 824 may be implemented by the mobile device 802.

The mobile device 802 may further include a wireless communication element 826. Further, the system 800 can include a wireless communication element 828 co-located within or otherwise integrated within the earphone or earbud. The wireless communication element 826 and the wireless communication element 828 may implement a wireless communication standard that allows the wireless communication element 826 and the wireless communication element 828 to wirelessly transmit communications between each other. For example, the wireless communication element 826 and the wireless communication element 828 may implement a Bluetooth communication standard in some embodiments. Further, the wireless communication element 826 and the wireless communication element 828 may each comprise a Bluetooth radio in some embodiments. The wireless communication element 826 may be coupled to the control system 822 and may exchange communications with the control system 822. The wireless communication element 828 may be coupled with the tone generator 804 and the SPL measurement element 820, and may provide communications to the tone generator 804 and receive communications from the SPL measurement element 820. Based on the wireless communication between the wireless communication element 826 and the wireless communication element 828, the control system 822 can communicate with the tone generator 804 and the SPL measurement element 820 via the wireless communication element 826 and the wireless communication element 828.

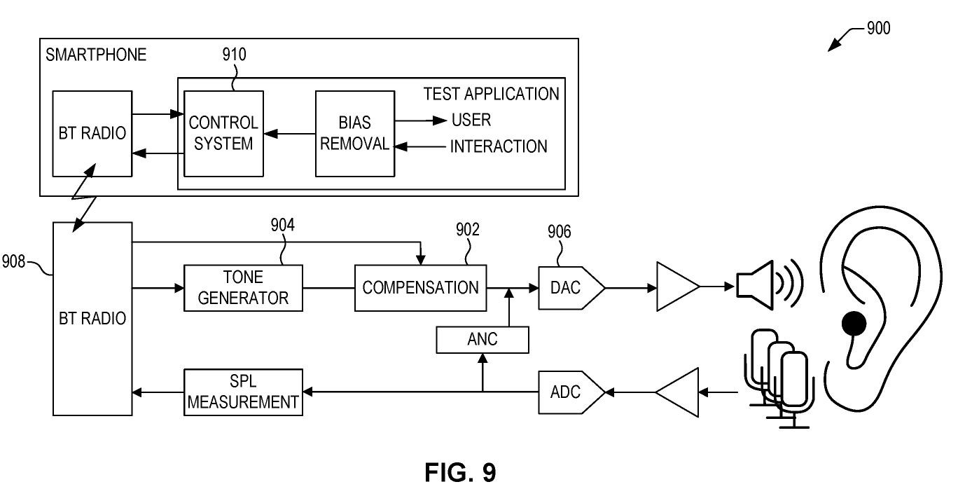

FIG. 9 illustrates an example system 900 that can implement the approaches described throughout this disclosure, according to some embodiments. For example, the system 900 may implement the hearing testing approaches, the unbiased testing approaches, and/or the compensation approaches described throughout this disclosure. The system 900 can be implemented in headphones, including an earphone or an earbud of the headphones, such as the earbud 1000 (FIG. 10). In some embodiments, the system 900, or some portion thereof, may be implemented in a processor located within the headphones. In some embodiments, a single earphone or earbud of the headphones may include the system 900, whereas each of the earphones or earbuds of the headphone may include the system 900 in other embodiments. The system 900 can include the same elements as the system 800 (FIG. 8).

The system 900 can further include a compensation element 902. The compensation element 902 may be coupled between a tone generator 904 and a DAC 906, where the tone generator 904 and the DAC 906 have the same features as the tone generator 504 (FIG. 5), and the DAC 506 (FIG. 5), respectively. Further, the compensation element 902 may be coupled to a wireless communication element 908 that can provide for communication between the compensation element 902 and a control system 910, where the wireless communication element 908 and the control system 910 have the features of the wireless communication element 828 (FIG. 8) and the control system 822 (FIG. 8), respectively. The compensation element 902 may receive communications from the control system 910 and utilize the communications to provide compensation to a signal received from the tone generator 904 and provided to the DAC 906 by the compensation element 902. The compensation that may be performed by the compensation element 902 may include performing a reverse equalization on the signal, apply a gain to frequencies within the signal corresponding to hearing loss of the user, lowering frequencies within the signal corresponding to good hearing of the user to match with frequencies corresponding to hearing loss of the user, and/or compression The compensation element 902 may be co-located within or otherwise integrated into the earphone or the earbud with other elements of the system 900 located within the earphone or earbud. In some embodiments, the IC or ICs that implement other elements of the system 900 located within the earphone or earbud can implement the compensation element 902.

FIG. 10 illustrates an example earbud 1000, according to various embodiments. Systems, or portions thereof, described throughout this disclosure can be implemented in the earbud 1000. For example, the system 500 (FIG. 5), the system 600 (FIG. 6), and the system 700 (FIG. 7) may be implemented in the earbud 1000 in some embodiments. Further, portions of the system 800 (FIG. 8) and the system 900 (FIG. 9) may be implemented in the earbud 1000 in some embodiments. A speaker (such as the speaker 510 (FIG. 5) or the speaker 812 (FIG. 8)) and a microphone (such as the microphone 512 (FIG. 5) or at least one microphone of the one or more microphones 814 (FIG. 8)) may be located within the earbud 1000 in some embodiments.

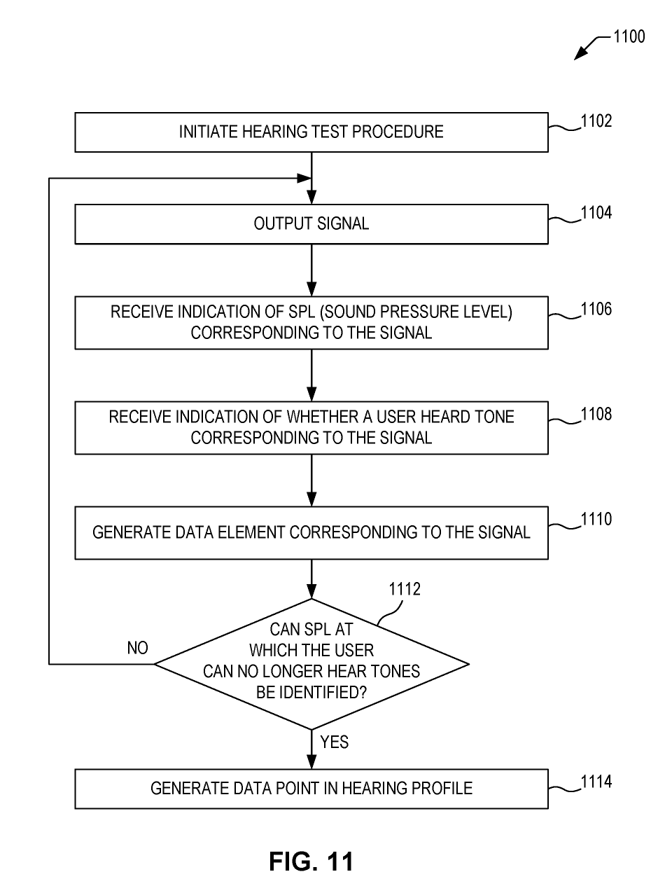

FIG. 11 illustrates an example hearing test procedure 1100, according to various embodiments. The hearing test procedure 1100 may be implemented by a control system of any of the control systems disclosed herein, such as the control system 502 (FIG. 5), the control system 704 (FIG. 7), the control system 822 (FIG. 8), and the control system 910 (FIG. 9).

In stage 1102, the hearing test procedure may be initiated.

In stage 1104, the control system may output a signal. The signal output by the control system may display characteristics of a signal that has not been output during the current hearing test procedure 1100. For example, the signal may have a different value, amplitude, frequency, timing offset, and/or voltage offset than signals previously output by the control system. The control system may determine the characteristics of the signal based on a predefined characteristic scheme or may be randomly determined, and the control system may generate the signal with the characteristics. In some embodiments, the predefined characteristic scheme may include increasing or decreasing one or more characteristics of the signal from the immediately preceding signal by a predefined amount. In some embodiments, the predefined characteristic scheme may include increasing or decreasing one or more of the characteristics of the signal based on whether the user indicated hearing the tone corresponding to the immediately preceding signal, where the amount of the change or changes can be determined based on one or more of the previous preceding signals.

In stage 1106, the control system may receive an indication of an SPL corresponding to the signal. The control system may receive the SPL from an SPL measurement element, such as the SPL measurement element 518 (FIG. 5), the SPL measurement element 610 (FIG. 6), and the SPL measurement element 820 (FIG. 8).

In stage 1108, the control system may receive an indication of whether the user heard a tone corresponding to the signal. The control system may receive user feedback indicating whether the user heard the tone corresponding to the signal.

In stage 1110, the control system may generate and store a data element corresponding to the signal. The data element may include an indication of the SPL corresponding to the signal and an indication of whether the user heard the tone corresponding to the signal.

In stage 1112, the control system can determine whether an SPL at which the user can no longer hear the tones can be identified. For example, the control system can determine whether a group of signals to be outputted for the hearing test procedure had been outputted by the control system and analyze the data elements corresponding to the signals to determine an SPL at which the user can no longer hear the tones. In other embodiments, the control system can analyze the data elements corresponding to the signals output by the control system in the current hearing test procedure and determine whether the SPL at which the user can no longer hear the tones can be determined within a predefined precision. The control system can determine the SPL at which the user can no longer hear the tones based on the indications of the SPLs and the corresponding indications of whether the user can hear the tones in the data elements. If the SPL at which the user can no longer hear the tones cannot be determined, the hearing test procedure 1100 can proceed to stage 1104. If the SPL at which the user can no longer hear the tones can be determined, the hearing test procedure 1100 can proceed to stage 1114.

In stage 1114, the control system may generate a data point in a hearing profile for the user based on the SPL at which the user can no longer hear the tones.

Technologies described in the present application can improve hearing tests beyond reducing the amount of calibration that may be necessary to perform a hearing test on a subject. More specifically, embodiments of the technologies described herein, individually or in combination, provide hearing tests and/or compensations that account for environmental factors and operation factors associated with a subject being tested. Accordingly, such embodiments may address several issues of existing hearing tests. For example, hearing measurements and/or headphone calibration are traditionally done in quiet environments under the assumption that speech in noise affects all subjects and recipients equally. As a result, subsequent compensations for hearing loss may not perform well in noisy environments, where multiple sound sources, including noise sources, may be present. Thus, subjects may need frequent re-fitting of their hearing devices.

Systems, devices, and methods in accordance with this disclosure, individually or in combination, permit implementing hearing tests that incorporate contextual information associated with the subject undergoing a hearing test or the environment where the hearing test is implemented. To that end, in some configurations, headphones may include one or more context-awareness elements that, individually and/or in combination, can obtain contextual information. For purposes of illustration, the context-awareness elements include modules and other types of components. Also for purposes of illustration, contextual information includes information defining environment context, information defining operation context, information defining operator context, or a combination of the foregoing types of information/contexts. Environment context includes, for example, acoustic characteristics of the environment, such as type and amount of ambient noise and/or position of the subject relative to a sound/noise source. Operation context includes, for example, an activity of the subject wearing a headphone in accordance with aspects of this disclosure, a time of day when the subject is wearing such a headphone, an amount of sound that has been played back by the headphones during a defined period, sound leakage present in the headphones, or the like. For purposes of illustration, the defined period may be eight hours, 12 hours, 24 hours, a work shift (as in a scenario where the headphone is worn in a call center or in another workplace), or the like, and the amount of sound that has been played during the defined period is referred to as “sound dose.” Operator context includes, for example, one or more physiological attributes of the subject, or anatomical characteristics of the subject, or a combination of the foregoing. As an illustration, a physiological attribute includes a biometric datum (a value of a vital sign, such as temperature, respiration rate, or hear rate; a value of SpO2; or the like), an indicator of presence of a health condition (e.g., a logical, string, or alphanumeric value indicating presence of a cardiac disorder, a cardiopulmonary disorder, or a neurological disorder), an indicator of a status of the health condition (e.g., a logical, string, or alphanumeric value indicating normal condition, exacerbated condition, unchanged condition, or improved condition), or similar attribute.

Reproducing the foregoing aspects of context during a hearing test can make the measurement significantly more robust by providing hearing results (e.g., audiograms) dependent on contextual information corresponding to an environment that surrounds the subject and/or to a condition (physical or emotional) of the subject themselves. The context awareness element(s) inside the headphone or a hearing aid device (or in some cases a lifestyle hearing device) can then be used to update hearing loss compensation continuously (e.g., as sound is received at the headphone or hearing aid device and an audio signal representative of the sound becomes available to the headphone or hearing aid device for processing) depending on the environment surrounding the subject, a physical condition of the subject, and/or an emotional condition of the subject.

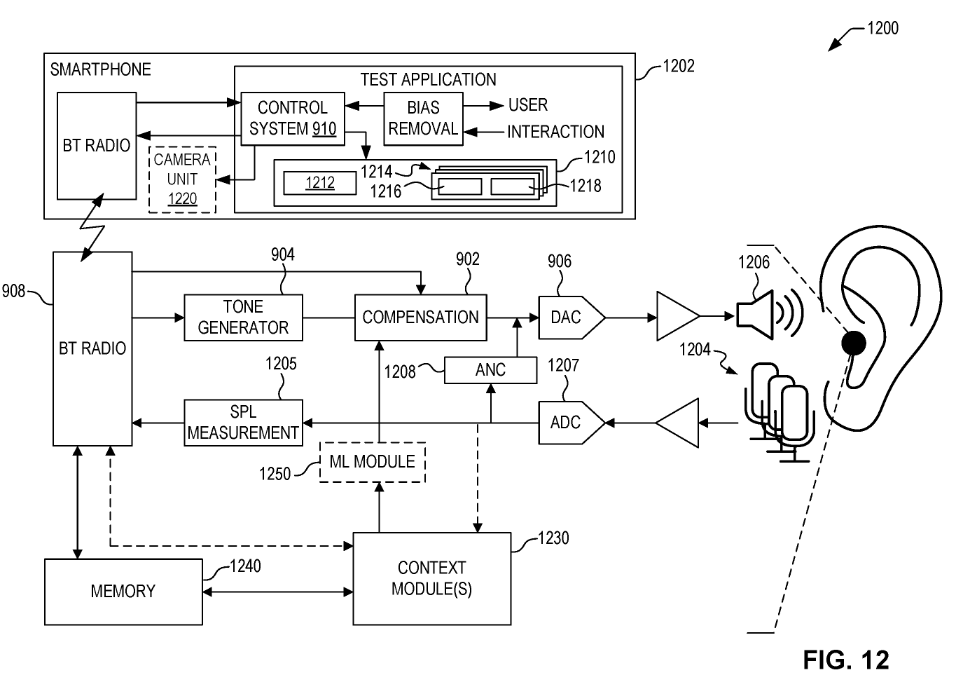

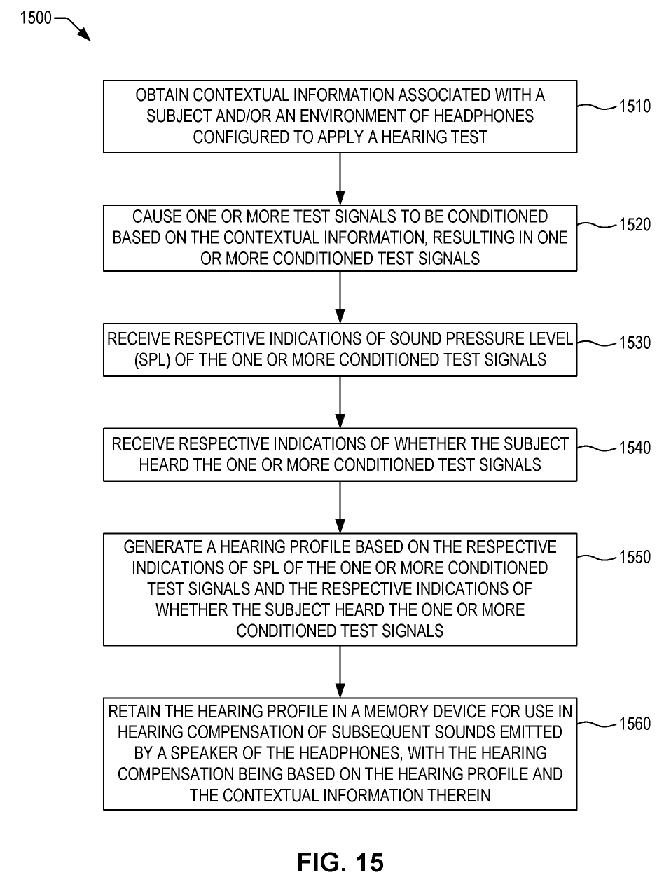

FIG. 12 illustrates an example system 1200 that can implement the approaches described throughout this disclosure, according to one or more aspects of this disclosure. The example system 1200 can apply a hearing test to a subject, via a mobile device 1202 and headphones worn by the subject. As in prior example systems, a headphone (such as an earbud) of the headphones is represented by a solid circle in FIG. 12. The hearing test can incorporate contextual information associated with the subject and/or an environment in which a hearing test is being performed. As is described herein, the contextual information includes, for example, at least one of physiological data of the subject, acoustic characteristics of the environment, or anatomical characteristics of the subject. The contextual information is represented by a block 1212 in FIG. 12 and can be retained in one or more memory devices 1210 (collectively referred to as memory 1210) of the mobile device 1202. The contextual information is herein referred to as contextual information 1212. The hearing test can be applied by the control system 910 in accordance with aspects of this disclosure. For example, the control system 910 can apply the hearing test by implementing the example method 1500 described herein and shown in FIG. 15. Although shown as being part of the mobile device 1202, the control system 910 may be part of the headphone in some cases.

The example system 1200, or at least the headphone portion thereof, can apply hearing compensation during routine use of the headphones (or a headphone device therein) as a hearing aid device or a lifestyle hearing device. The hearing loss compensation is based on contextual information associated with the subject and/or an environment in which the headphones are used as a hearing aid device or a lifestyle hearing device.

The example system 1200 includes one or multiple context modules 1230 that, individually or in combination, can obtain contextual information and can supply the contextual information to the control system 910. Each of the context module(s) 1230 can be implemented as hardware, software, or a combination of hardware and software. In some cases, a particular context module of the context module(s) 1230 can obtain contextual information actively. That is, is the control system 910 can direct or otherwise cause the particular context module to obtain, e.g., measure, a particular type of contextual information. In addition, or in other cases, another particular context module of the context module(s) 1230 can obtain contextual information passively. That is, that another particular context module can obtain a particular type of contextual information without a command or another type of indication to obtain contextual information. The another particular context module can be responsive to an environment of the headphone (and the subject using the headphone) and/or a condition of the subject, generating measurement data defining, at least partially, the contextual information. To that end, the another particular context module may include one or more transducer devices that can generate, individually or in combination, the measurement data. In one example, the another particular context module includes a gyroscope that permits measuring changes in orientation of the head of the subject wearing the headphone, thus obtaining operator context. In another example, the another particular context module includes an electronic thermometer the permits measuring temperature of the subject wearing the headphone, thus obtaining operator context. In yet another example, the another particular context module includes positioning information, such as GPS, that permits adjusting the hearing test and/or the results of the hearing test, and also predicting and preparing the headphone to transition to a different mode of operation. In still another example, the another particular context module includes other physiological information about the subject wearing or prepared to wear the headphone, such as glycemia, electrocardiogram (ECG), electromyogram (EMG), electrooculogram (EOG), electroencephalogram (EEG), heart rate variability, stress level, sleeping score, and/or similar physiological information.

Contextual information can be used for implementation of a hearing test. As mentioned, the hearing test can be implemented in accordance with aspects described herein. To incorporate contextual information, in some cases, the hearing test includes repeating testing for a subject under a particular context. Such a context is defined by contextual information and may include particular operation context, operator context, and/or environment context. As a result, a hearing test can yield multiple hearing profiles, each including data defining an audiogram—that is, a result of an iteration of testing at the particular context—and metadata defining the particular context for the audiogram. In FIG. 12, the multiple hearing profiles are represented by blocks 1214, and the data and metadata are respectively represented by block 1216 (herein referred to as audiogram 1216) and block 1218 (herein referred to as context 1218). After the hearing test has been implemented, the control system 910 can retain the hearing profiles in one or more memory devices 1240 (collectively referred to as memory 1240) integrated into the headphone included in example system 1200. The memory 1240 includes at least one non-volatile memory device.

A hearing test that incorporates contextual information in accordance with aspects of this disclosure is contextualized in various ways. For example, the hearing test is personalized to the subject being tested or about to be tested and/or customized to an environment where the subject may use headphones fitted according to the hearing test.

In addition, contextual information also can be used to apply hearing compensation during routine use of the headphones as a hearing aid device or a lifestyle hearing device, after a hearing test that has included the contextual information. Thus, the headphones in accordance with aspects of this disclosure can provide contextualized hearing compensation that is personalized to the subject wearing or prepared to wear the headphones and/or customized to an environment where the headphones are utilized. That is, a context module of the context module(s) and/or another type of module present in the headphones can apply a hearing compensation (or hearing enhancement) profile that is consistent with environmental, operational, and/or operator conditions. Accordingly, such modules can cause the headphones to apply an adequate compensation profile, and, thus, provide consistently satisfactory hearing in most, if not all, situations.

By contextualizing hearing tests and utilization of headphones or a headphone device (e.g., an earbud) used as hearing aid devices or lifestyle hearing devices, the hearing tests and headphones (or headphone devices) in accordance with aspects of this disclosure are improved over existing hearing tests and hearing aid devices. For example, the rich information that constitutes the contextual information described herein provides superior hearing tests that characterize hearing under myriad conditions. This stands in sharp contrast to fitting of hearing aid devices traditionally performed by an audiologist in a control environment where only a few scenarios may be accounted for. As another example, utilization of headphones that incorporate the contextual information described herein can adapt hearing compensation to myriad conditions. Thus, headphones in accordance with aspects of this disclosure flexibly provide hearing compensation that ensures adequate hearing health consistent with a contextualized hearing test of a subject, without the need to repeat hearing tests or refitting operations in response to changes in personal conditions and/or environmental conditions of the subject.

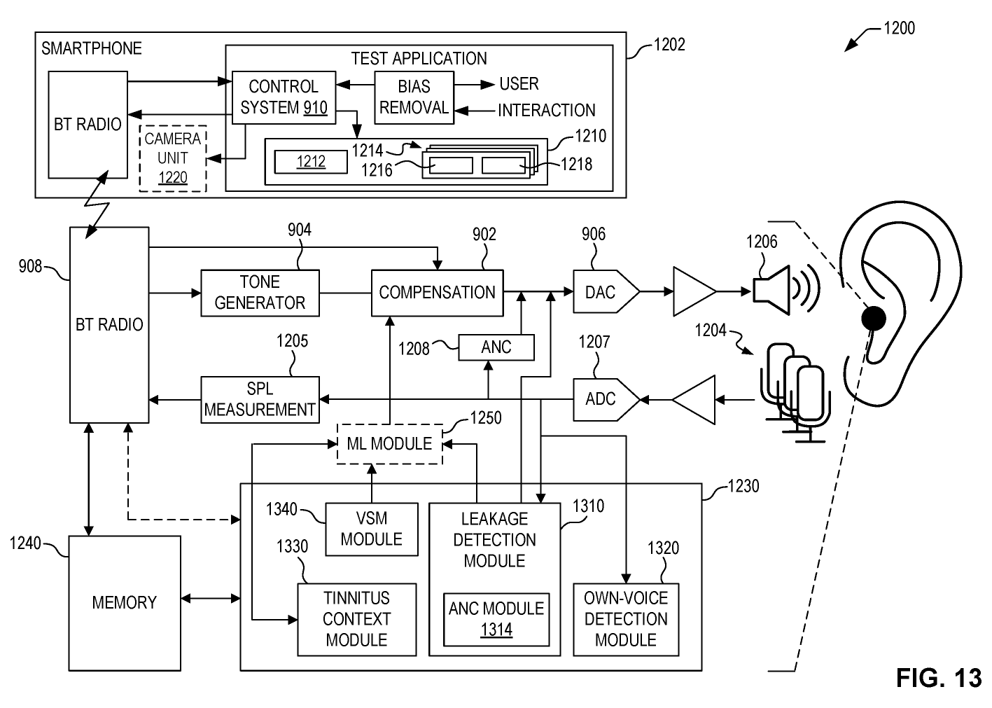

To illustrate the various types of contextual information that the context module(s) 1230 can obtain, FIG. 13 presents an example of context module(s) 1230 that can be integrated into the example system 1200, according to one or more aspects of this disclosure. As is shown in FIG. 13, the context module(s) 1230 include a leakage detection module 1310 that can detect sound leakage passing through the headphones.

Sound leakage may arise when headphones (or earbuds) do not form a perfect seal around or within the ears of the subject wearing the headphones. As a result of sound leakage external ambient noise/sound is allowed to enter the headphones and internal test signals (e.g., tones) are allowed to escape the headphones. Sound leakage can be caused by a variety of factors, including poorly fitted earbuds, headphones worn with eyeglasses, hats, or other accessories that prevent a tight acoustic seal.

The leakage detection module 1310 includes, in some cases, an ANC module 1314 that can detect, identify, and/or compensate for an amount of sound leakage passing through the headphones, depending on how the headphones are worn or inserted in or around the ears. The ANC module 1314 can implement adaptive ANC in some configurations. The ANC module 1314 and at least one of microphone(s) 1204 form a noise-cancellation subsystem within the example system 1200. In some cases, the microphone(s) 1204 form a microphone array having multiple microphones distributed over and/or within the headphones. Thus, in some configurations, the ANC module 1314 uses one or multiple microphones of the microphone array as a feedback microphone array to implement feedback-only ANC. In other configurations, the ANC module 1314 uses one or multiple microphones of the microphone array as a feed-forward microphone array and implements feed-forward-only ANC. The feed-forward microphone array may be disposed in the headphone/earbud so as to receive audio signal (e.g., pressure waves) from the exterior of the ear. In yet other configurations, the ANC module 1314 uses first and second microphone arrays of the microphone array as the feedback path and the feed-forward path, and can implement hybrid ANC.

The leakage detection module 1310, via the ANC module 1314, can continuously monitor a response signal from a feedback microphone (or, in some cases, a feedback microphone array) inside the ear canal of a subject and can adjust noise cancellation filters accordingly to compensate for a detected amount of sound leakage. In this way, the ANC module can maintain a consistent ANC response and a resulting stable sound output at the headphones. For purposes of illustration, a consistent ANC response includes a nearly constant amplitude of an audio signal, a desired level of signal-to-noise ratio (SNR), or the like. As such, in situations where the earbud/headphone begins or continues to slip during a hearing test—due to, for example, movement, sweating, or yawning—the adaptive ANC process compensates to maintain a consistent ANC response so that the accuracy of the subject’s responses to test tones is not compromised by the sound leakage. In some configurations, the feedback microphone (or feedback microphone array) is part of the microphone(s) 1204. In other configurations, the feedback microphone (or feedback microphone array) is a dedicated microphone (or microphone array) that can be used for purposes of sound leakage monitoring and can supplement audio signal generated by the one or more microphones 1204.

To detect an amount of sound leakage, the leakage detection module 1310, via the ANC module 1314, can continuously monitor an ambient audio signal from a feed-forward microphone (or a feed-forward microphone array) disposed on the headphone/earbud so as to receive ambient sounds. The feed-forward microphone (or a feed-forward microphone array) can be part of the microphone(s) 1204. By determining a residual signal defined as a response signal relative to the ambient audio signal, the leakage detection module 1310, via the ANC module 1314, can detect the amount of sound leakage. Here, the response signal is determined by the ANC module 1314 continuously in response to the ambient audio signal (which is a temporal signal that may change over time or may remain nearly unchanged). In the absence of sound leakage, the response signal is a 180^{o} out-of-phase target copy of the ambient sound signal.

Because the hearing test is performed, in part, via the mobile device 1202 (e.g., a smartphone) there may be no restrictions on the movement of the subject undergoing the hearing test. Accordingly, the amount of sound leakage that is detected by the leakage detection module 1310 can change over time as a result of movement of the headphones, changes in fit of the headphones relative to the ear of the subject, and/or an accessory worn by the subject near the ear of the subject. Regardless of changes in the amount of leakage, the leakage detection module 1310, via the ANC module 1314, can maintain a stable audio output in the headphones.

After the hearing test has been completed, the leakage detection module 1310 can provide, via the compensation element 902, stable audio output in the presence of sound leakage. Specifically, an amplitude, e.g., SPL, of the stable audio output can be consistent with a desired amplitude based on the hearing test. In other words, the leakage detection module 1310 can provide, via the compensation element 902, appropriate audio performance after the hearing test, without a need for re-testing (as would be the case in existing technologies that do not incorporate sound leakage in hearing tests.

Contextual information including an amount of sound leakage may be used in conjunction with adaptive filtering, machine learning and/or artificial intelligence (AI) to identify patterns between sound-leakage conditions and hearing performance, enabling the example system 1200 to dynamically update hearing compensation settings/parameters based on a current amount of sound leakage. More specifically, a machine-learning model can be trained using at least some hearing profiles of the multiple hearing profiles 1214, with that subset of the multiple hearing profiles 1214 obtained at different respective amounts of sound leakage. Such a training results in a machine-learned model that can map an amount of sound leakage to one or more hearing compensation settings/parameters for satisfactory hearing performance of the subject wearing the headphones.

In some configurations, such a dynamical update may be performed using a machine-learning module 1250 (ML module 1250). The ML module 1250 can be implemented as hardware, software, or a combination of hardware and software. The machine-learning module 1250 includes the machine-learned model (not depicted) that maps an amount of sound leakage to one or more hearing compensation settings/parameters predicted to provide an adequate hearing performance in the presence of the amount of sound leakage. The ML module 1250 can supply the one or more hearing compensation settings/parameters to the compensation element 902. In response, the compensation element 902 can configure an appropriate level of hearing compensation. Although the ML module 1250 is shown as being external to the context module(s) 1230, the disclosure is not limited in that respect and the ML module 1250 may be part of the context module(s) 1230.

As is also shown in FIG. 13, the context module(s) 1230 also can include an own-voice detection module 1320 (OVD module 1320). The OVD module 1320 can determine that, at a time or time interval, a subject is speaking, coughing, or otherwise producing vocalizations. The subject may be undergoing a hearing test. As such, the OVD module 1320 can detect an utterance or another type of vocalization produced by the subject during the hearing test.

To detect an utterance or another type of vocalization, the OVD module 1320 can include or can be coupled with at least one feedback microphone located inside an ear canal of the subject. The at least one feedback microphone can be one or multiple ones of the microphone(s) 1204. In other configurations, instead of including such dedicated feedback microphone(s), the OVD module 1320 can use audio signal generated by the microphone 1204. In addition, or in yet other configurations, instead of including or relying upon a feedback microphone, the OVD module 1320 can include a bone vibration sensor device co-located within or otherwise integrated into the headphones, in an earphone or an earbud of the headphones, for example. The bone vibration sensor device is an accelerometer device (such as a multi-axis MEMS accelerometer) in some cases. Such a sensor device may be part of the OVD module 1320 or may be coupled therewith.

The feedback microphone(s) that may be part of or may be coupled with the OVD module 1320 can capture the occlusion effect—a “booming” sound that occurs when the ear is sealed and an utterance is emitted—which is characteristic of the subject’s own voice. In contrast, in configurations where the OVD module 1320 includes or is coupled with a bone vibration sensor device, the bone vibration sensor device can measure physical vibrations generated by utterances, speech, and/or other vocalizations. The bone vibration sensor device is substantially immune to external acoustic noise.

Own-voice detection serves at least two purposes in the example system 1200. First, it adds robustness to a hearing measurement by allowing the example system 1200 to reject and repeat any test measurements taken while the user was speaking, coughing, or otherwise producing sounds that could interfere with accurate results. Second, it enables a voice user interface, allowing the user to provide an aural response (e.g., indicating whether a tone was heard) rather than pressing a physical button, thereby improving accessibility. The use of the bone vibration sensor in conjunction with the feedback microphone makes OVD reliable even in very noisy environments.

As is further shown in FIG. 13, the context module(s) 1230 also can include a tinnitus context module 1330 that can identify a tinnitus status of a subject undergoing a hearing test or wearing the headphones after the hearing test. Simply for purposes of illustration, tinnitus is described as a condition in which the cochlea produces phantom sounds—typically tones at certain frequencies—that the subject perceives in their ears. In cases where the subject is afflicted by tinnitus, the tinnitus context module 1330 can supply data identifying the presence of tinnitus and/or frequency information corresponding to the subject. The frequency information includes data defining one or more tinnitus frequencies corresponding to the subject. The tinnitus context module 1330 can supply such data to the control system 910 in response to performance of a hearing test, as a result of the hearing test. During use of the headphones as a hearing device, the tinnitus context module 1330 can supply the data defining one or more tinnitus frequencies to the compensation element 902.

In some cases, the control system 910 configures the headphones, at least partially, by providing the data defining the one or more tinnitus frequencies to the tinnitus context module 1330. The control system 910 can receive user-input information indicative of the tinnitus frequencies of the subject undergoing a hearing test or using the headphones as a hearing aid device or lifestyle hearing device. The user-input information can be received via user-interaction between the subject and the mobile device 1202 configured with the test application to perform the hearing test.

Tinnitus is not constant; it can vary in intensity depending on various factors, such as the subject’s health conditions (e.g., chronic or temporary hypertension), fatigue level, stress level, time of day, and/or current sound dose, for example. Thus, the tinnitus context module 1330 also can send data indicating that the subject wearing the headphones is experiencing tinnitus at a time or time interval during the day, based on one or more of those factors. Such data can be sent, during the hearing test, to the compensation element 902 that is integrated into the headphone in the example system 1200.

In some cases, the tinnitus context module 1330 determines a tinnitus state of the subject wearing the headphones. The tinnitus state may be one of “tinnitus present” or “tinnitus absent,” where tinnitus present indicates that the subject is experiencing tinnitus and tinnitus absent indicates that the subject is not experiencing tinnitus. To that end, the tinnitus context module 1330 can apply one or more rules to the factors mentioned above, including subject’s health conditions (e.g., chronic or temporary hypertension), fatigue level, stress level, time of day, and/or current sound dose, for example.

In other cases, the tinnitus context module 1330 requests the ML module 1250 to determine the tinnitus state of a subject wearing the headphone and a confidence level of tinnitus being present or absent. The ML module 1250 can determine the tinnitus state and confidence level by applying a binary-task classification model (not depicted in FIG. 13) to the factors mentioned above, including subject’s health conditions (e.g., chronic or temporary hypertension), fatigue level, stress level, time of day, and/or current sound dose, for example. Such a classification model is a decision tree in some cases.

The compensation element 902 can receive data from the tinnitus context module 1330, where, as is described hereinbefore, at least part of the data can be indicative of the tinnitus state of a subject wearing the headphones in the example system 1200. In response to receiving the data, the compensation element 902 can condition test signals and/or other audio signals accordingly. For instance, during a hearing test, the compensation element 902 can condition one or more test signals by adjusting the one or more test signals to avoid having respective frequencies overlapping tinnitus frequencies corresponding to a subject.

By shifting the test tones outside a frequency range including the tinnitus frequencies or by playing tones louder to account for the masking effect of tinnitus, the control system 910 can implement a hearing test that is robust relative to tinnitus. Results of the hearing test also include metadata representative of tinnitus presence and associated tinnitus frequencies. Specifically, the hearing test results in a hearing profile that includes an audiogram 1216 and metadata 1218 representative of, among other context, presence of tinnitus and tinnitus frequencies.

Additionally, accurate tinnitus information is important for proper hearing compensation, because improperly boosting frequencies in the tinnitus region could cause discomfort or even further damage to the hearing of a subject. The example system 1200, via an earbud or earphone of the headphones, can implement a relationship between tinnitus and vital sign monitoring (VSM). Accordingly, the headphones can mitigate, or avoid altogether, the exacerbation of tinnitus intensity when a subject using the headphones is tired or in poor health.

As is further shown in FIG. 13, the context module(s) 1230 also can include a VSM module 1340 that can obtain contextual information that includes biometrics data. The VSM module 1340 includes one or more VSM sensor devices integrated into or coupled with the headphones. The one or more VSM sensor devices can be configured, individually or collectively, to generate measurement signals that either represent or can be processed to define one or more physiological attributes of a subject. The physiological attributes include, for example, heart rate, respiratory rate, temperature, and/or a value of peripheral blood oxygen saturation (SpO2). The VSM module 1340 can process at least some measurement signals, such as photoplethysmography (PPG) signals, to determine heart rate, SpO2, or similar biometric quantities.

In the same fashion that tinnitus intensity (e.g., the amplitude of tinnitus sound perceived by a subject afflicted by tinnitus) can vary depending on health status and physical condition of a subject at a time a hearing test is performed or as the subject wears the headphone/earbud after the hearing test is complete, the hearing abilities of the subject also depend on health status and physical condition of the subject. For example, a subject who is stressed, fatigued, and/or has been exercising may hear differently than when rested and/or calm. Thus, these physical conditions can bias or otherwise distort the results of a hearing test. By obtaining vital signs alongside hearing test data (e.g., an audiogram), the example system 1200 (FIG. 12) can contextualize and calibrate the measurement results to improve accuracy and robustness. That is, as is described herein, the example system 1200, via the control system 910, for example, can generate a hearing profile that includes both an audiogram resulting from a hearing test and metadata indicative or otherwise representative of vital sign(s) and/or other biometric data (e.g., cortisol level).

Contextual information including vital sign data and/or other physiological data also may be used in conjunction with machine learning and/or artificial intelligence (AI) to identify patterns between physiological states and hearing performance, enabling the example system 1200 to dynamically update hearing compensation settings/parameters based on a current physical state and/or emotional state of a subject. For instance, if the example system 1200 detects that a subject is tired (e.g., the subject has low level of SpO2 or bradycardia) or has elevated heart rate, the example system 1200, via one or a combination of components integrated thereto, may adjust a compensation profile of a headphone accordingly. The headphone may be used as a hearing aid device or lifestyle hearing device. As such, applying hearing compensation may include dynamically updating, by the compensation element 902, the hearing compensation based on subsequent measurements of one or more physiological attributes. Hence, the example system 1200 can provide hearing compensation that is personalized to a subject and/or can promote hearing health of the subject.

In some configurations, such a dynamical update may be performed using the ML module 1250 that may be included in the context module(s) 1230. The ML module 1250 includes a machine-learned model (not depicted) that can correlate the at least one physiological attribute with hearing performance of the subject wearing the headphones. More specifically, the machine-learned model has been trained using at least some of the multiple hearing profiles 1214 to map at least one physiological attribute to one or more hearing compensation settings/parameters for satisfactory hearing performance of the subject wearing the headphones. The ML module 1250 can supply the one or more hearing compensation settings/parameters to the compensation element 902. In response, using the one or more hearing compensation settings/parameters, the compensation element 902 can configure an appropriate level of hearing compensation.

Another form or contextual information involves spatial rendering. The example system 1200 (FIG. 12), via the tone generator 904, can generate such contextual information by generating audiograms or other types of hearing test results using preconditioned test signals (e.g., tones) with spatial rendering based on head-related transfer functions (HRTFs). For purposes of illustration, without intending to be bound by theory and/or modeling, HRTFs characterize how sound is shaped by the unique physical anatomy of a subject—including nose, cheekbones, ear shape, and head size—before reaching the eardrum, enabling the brain to perceive sound as coming from specific directions in three-dimensional space. In typical headphone playback, sound is perceived as originating from the middle of the head, which does not reflect real-world hearing conditions. That is, after a hearing aid device (such as headphones) is configured/fitted using a hearing test for a subject, a subject wears the hearing aid device in environments where sound sources may be dispersed around the subject, emitting sound from different directions.

To generate contextual information involving spatial rendering, for example, the control system 910 can cause the mobile device 1202 (e.g., a smartphone) to capture an HRTF of a subject. In response, the mobile device 1202 can direct the subject to use the mobile device 1202 to scan ears, face, and/or head of the subject, via a camera unit 1220 or depth sensor device (not depicted) integrated into or coupled with the mobile device 1202. The mobile device 1202 can direct the subject to scan ears, face, and/or head of the subject by presenting an aural prompt (e.g., a particular sound, a ringtone, or recorded voice command, or a synthetic voice command), visual prompt, and/or a haptic prompt (e.g., a vibration or patter of vibrations). Regardless of how the mobile device 1202 directs/prompts a subject to perform such a scan, in some cases, the scan involves positioning the mobile device 1202 at multiple angles relative to a line of sight of the subject, with the camera unit 1220 facing the subject, and then directing/prompting the subject to move the subject’s head to generate imaging data representing the anatomy of the head, face, and ears of the subject.

The control system 910 can then use the acquired imaging data to generate the HRTF. Specifically, as described above, the acquired imaging data defines a three-dimensional (3D) scan (or 2D with extra processing) of the ears, face, and/or head of the subject. The 3D scan can be processed to generate a 3D mesh model of the 3D scan. In some cases, the 3D scan can be transformed into a two-dimensional (2D) scan, which 2D scan is used to generate a 2D mesh model. An acoustic model can then be generated using both the 3D mesh model (or in some cases, the 2D mesh model) and multiple defined virtual sound sources distributed in space. Generating the acoustic model in such a fashion includes simulating sound propagation in the presence of 3D mesh model to simulate the SPL of sounds based on angles and/or positions. The acoustic model can be generated using, for example, a boundary element method (BEM) or fast multipole BEM. Then the acoustic model is transformed (via an inverse fast Fourier transform (iFFT), for example) into a head-related impulse response (HRIR) that can be formatted into a filter that can be embedded into a headphone or another type of hearing device and used via, for example, convolution to generate a desired spatial rendering or to cause the generation of the desired spatial rendering. The filter can be embedded/retained in a non-volatile memory device or devices of the memory 1240 of the headphone.

The camera unit 1220 is shown as an optional element because, in some cases, also to generate contextual information involving spatial rendering, the control system 910 can use a preconfigured HRTF instead of capturing/generating the HRTF of a subject. The preconfigured HRTF can be used for any subject that wears headphones according to aspects of this disclosure for either a hearing test or routine hearing loss compensation. The contextual information 1212 retained in the memory 1210 can include the preconfigured HRTF.

Simply for purposes of illustration, regardless of how an HRTF is generated, the HRTF represents a spatial acoustic filter that models spatial cues corresponding to a subject or a model of subject (e.g., a dummy or mannequin). The spatial cues, individually and in combination, affect how sound is propagated to the ear canal of the subject (or the model of the subject) and include face shape, ear shape, amount of hair, size and shape of nasal and oral cavities, and the like. Thus, the spatial cues cause each person to hear in slightly different manners. The auditory system and brain of the subject decode sound according to the HRTF of the subject.

The HRTF contains information on sound source location based on various cues: Interaural time difference (ITD), interaural level difference (ILD), and pinna reflections. The ITD is the time difference of sound arrival between left and right ears. Unless sound arrives precisely from the front of the subject or from the back of the subject, with a strictly symmetrical head, a sound wave can reach both ears at slightly different times. Similarly, the level (or amplitude) of a sound wave may not be the same at both ears because the head itself creates an acoustic “shadow” so a sound wave from the left can arrive with a higher level at the left ear than at the right ear or vice versa. The difference between such levels (amplitudes) is referred to as the ILD. Pinna reflections (or outer-ear shape reflections) of sound are unique to each subject because subjects have different ear shapes. Pinna reflections occur when a sound wave reaches an ear, before all the frequencies of the sound wave reach the ear canal and the tympanic membrane.

Besides other factors, sound localization is primarily based on ITD, ILD, and pinna reflections. Specifically, ITD and ILD allow the brain to decode range (distance) and lateral localization of an incoming sound wave, e.g., sound from left side, sound from right side, and distance. The pinna reflections permit decoding elevation, front/back localization, and finer placement.

The control system 910 can send data defining an HRTF to the tone generator 904 present in the headphones. In other configurations, the control system 910 can send the data defining the HRTF to the memory 1240 and the tone generator 904 can obtain the data from the memory 1240. The HRTF either has been personalized for a particular subject or is preconfigured. The tone generator 904 can then use the HRTF to condition test tones (or another type of test signal) by pre-filtering the test tones (or the other type of test signal) to simulate spatial sound perception, accounting for the unique anatomy of the head and ears of a subject undergoing a hearing test or prepared to undergo the hearing test. To that end, in some cases, the control system 910 generates a tone signal configured in the form of spatial audio, such as an Ambisonics signal formatted in B-format, and sends the tone signal to the tone generator 904. Although a particular format is mentioned herein, the disclosure is not limited to that format, and other formats for spatial audio also can be used in accordance with aspects of this disclosure. In response to receiving the tone signal, the tone generator 904 applies the HRTF to one or more audio channels (e.g., four channels in B-format) used to represent the tone signal. The channels can be represented as a polynomial, for example, defined as an expansion in series of spherical harmonics. By pre-filtering tests tones (or other types of test signals), a headphone of the example system 1200 can render sound as arriving from various directions, including from the front, back, and/or sides of the subject, and/or at different elevations relative to the head of the subject.

The inclusion and application of an HRTF to a hearing test can permit the tone generator 904 to position, virtually, a source of a tone in space at a particular position relative to the subject undergoing the test. Accordingly, the headphones of the example system 1200 can reproduce tones beyond monophonic playback and stereophonic playback. Thus, the example system 1200 and the headphones included therein, individually or in combination, clearly improve the functioning/operation of existing computerized systems for hearing testing.

By sweeping through various directions, the example system 1200 can apply a spatial hearing test. Indeed, the tone generator 904 can be caused to apply a hearing test that can be, by construction, a 360-degree hearing test that more closely replicates real-life listening conditions. Such a 360-degree hearing test, or a spherically sectorized version of such a hearing test, may yield more accurate and predictive hearing test results compared to commonplace left-right-only hearing testing. Additionally, since hearing impairment is often asymmetric between the two ears of a subject, a hearing test that involves spatial audio testing (such as a sectorized version of a 360-degree hearing test) can reveal how the brain compensates, between the ears, for sounds arriving from different angles relative to the subject undergoing the hearing test. Spatial audio also can be used in conjunction with ANC, via an ANC element 1208 in the example system 1200, where data defining the HRTF can permit the ANC element 1208 to focus noise cancellation in the specific direction from which sound (e.g., speech, utterances, noise, and the like) is arriving. The ANC element 1208 can operate at least in the same fashion as any other ANC element described herein operate, such as the ANC element 614 (FIG. 6) or the ANC element 806 (FIG. 8).

A hearing test that uses spatial rendering via an HRTF can provide a more natural experience to the subject undergoing the hearing test because test tones can be played-back (or reproduced) from multiple directions, as opposed to the traditional left-right testing. By configuring the test tones (or other types of test signal) to appear as arriving from multiple directions, the headphone of the example system 1200 can cause the hearing test to be perceived as being conducted as if the subject under testing is not wearing a sound playback device. Similarly, captured sound to be compensated for hearing loss also can be reproduced in surround sound, via Ambisonics, for example. The captured sound can thus be reproduced more naturally, as if the subject is not wearing the headphones included in the example system 1200.

Although the example system 1200 that is shown in FIG. 13 includes the leakage detection module 1310, the OVD module 1320, the tinnitus context module 1330, and the VSM module 1340, the disclosure is not limited to that particular configuration of context modules. Indeed, in other configurations, the example system 1200 can include fewer context modules that those context modules, in any particular combination. The number and type of context modules present in those other configuration may be dictated by form factor, target complexity, target energy efficient of the headphones, and/or other factors.

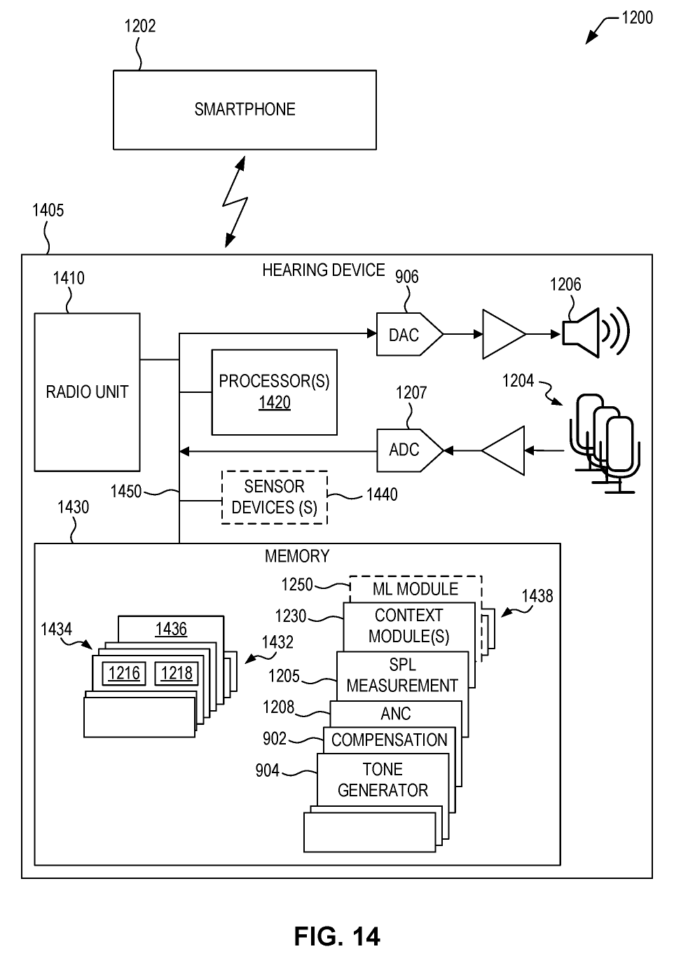

As mentioned, the optional ML module 1250 and each of the context module(s) 1230 can be implemented as hardware, software, or a combination of hardware and software. FIG. 14 illustrates an implementation of the example system 1200 where the context module(s) 1230 are implemented, at least partially, as software. More specifically, as is described herein, the example system 1200 that is shown in FIG. 14 includes the mobile device 1202 and a hearing device 1405 that corresponds to the headphone represented by a solid circle in FIGS. 12-13. In addition to various functional elements described herein, the hearing device 1405 includes a radio unit 1410, one or more processors 1420, one or more memory devices 1430 (collectively referred to as memory 1240), and optionally, one or more sensor devices 1440. The radio unit 1410 permits sending and receiving data and signaling wirelessly to and from an external device, such as the mobile device 1202. The radio unit 1410 sends and receives data according to one or more radio technology protocols (Wi-Fi, Bluetooth, etc.). In one example, the radio unit 1410 is or includes the wireless communication element 908. The memory 1430 includes at least one non-volatile memory device and is or includes the memory 1240.

The hearing device 1405 shown in FIG. 14 also includes a bus architecture 1450 that can communicatively couple at least two of the radio unit 1410, the processor(s) 1420, the memory 1430, or the sensor device(s) 1440 (in configurations that include such sensor device(s)). The bus architecture 1450 also can communicatively couple at least one of such functional elements with the DAC 906 and/or the ADC 1207. The bus architecture 1450 can permit exchanging data and signaling between two or more of such functional elements. Although not shown, the hearing device 1405 also includes a rechargeable battery or another type of power supply.

The memory device(s) 1430 include data 1432 and processor-accessible instructions 1438. The data 1432 includes one or more hearing profiles 1434, each one including an audiogram 1216 and metadata 1218. In some cases, a single hearing profile constitutes the hearing profile(s) 1434, with the single hearing profile corresponding to a subject for which the hearing device 1405 has been fitted according to a hearing test. The hearing test may be implemented as is described herein (in accordance with the method shown in FIG. 14, for example). In other cases, for example in scenarios where different subjects can use the hearing device 1405, multiple hearing profiles can constitute the hearing profile(s) 1434. Each one of multiple hearing profiles may correspond to a respective subject. Each one of the hearing profile(s) 1434 can be used for sound compensation in accordance with aspects described herein.

The data 1432 also includes contextual information 1436. In accordance with aspects described herein, the contextual information 1436 includes, for example, at least one of physiological data of the subject, acoustic characteristics of the environment, or anatomical characteristics of the subject. Further, the data 1432 also can include data defining one or more machine-learning models that may used by the ML module 1250 when present in the hearing device 1405.

The processor-accessible instructions 1438 include processor-readable instructions and/or processor executable instructions. The processor-accessible instructions 1438 can be arranged as modules and/or other types of components that provide functionality to the hearing device 1405, in accordance with aspects described herein. The modules can include, for example, one or a combination of tone generator 904, compensation element 1208, ANC element 1208, SPL measurement element 1205, context module(s) 1230, and optionally the ML module 1250. Although FIG. 14 illustrates all the foregoing modules as constituting the processing-accessible instructions 1438, the disclosure is not limited in that respect.

The context module(s) 1230 retained in the memory 1430 can include one or a combination of the leakage detection module 1310, OVD module 1320, tinnitus context module or VSM module 1340. Accordingly, in some cases, at least one sensor device of the optional sensor device(s) 1440 can form part of at least one context module of the context module(s) 1230. In one example, part of the VSM module 1340 is retained in the memory 1430 and part of the VSM module 1340 is formed by one or more of the optional sensor device(s) 1440. In addition, or in another example, part of the OVD module 1320 is retained in the memory 1430 and part of the OVD module 1320 is formed by an accelerometer device that is part of the optional sensor device(s) 1440. Further, or in yet another example, part of the leakage detection module 1310 is retained in the memory 1430 and part of the leakage detection module 1310 is formed by at least one of the microphone(s) 1204.

Regardless of the particular configuration/composition of the processor-accessible instructions 1438, the functionality of the hearing device 1405 shown in FIG. 14, in accordance with aspects described herein, can be provided in response to execution of at least a portion of the processor-accessible instructions 1438 by the processor(s) 1420 individually or in a particular combination. That is, the processor(s) 1420, individually or in the particular combination, can execute at least one the tone generator 904, compensation element 1208, ANC element 1208, SPL measurement element 1205, context module(s) 1230, or optionally the ML module 1250, to provide the functionality described herein in connection with implementation of a hearing test or use of the hearing device 1405 after a hearing test has been performed. More specifically, execution of at least a portion of the processor-accessible instructions 1438 by the processor(s) 1420, individually or in a particular combination, causes the hearing device 1405 to implement a hearing test and/or be used post-testing as a hearing aid device or lifestyle hearing device. Both the hearing test and post-testing use can incorporate contextual information associated with a subject that has undergone the hearing test and/or associated with an environment in which the hearing test is performed or the subject uses a hearing device 1405 configured according to results of the hearing test.

Execution of the processor-accessible instructions 1438 can involve generation and/or use of at least a portion of the data 1432. To that end, at least some of the data 1432 can be retained in processor-accessible data structures, such as registers, files, libraries, and the like. For example, execution of at least some of the modules that are part of the processor-accessible instructions 1438 can involve generation of the contextual information 1436. In addition, execution of at least some of the modules can include use of at least a portion of the contextual information 1436.

Example methods that can be implemented in accordance with this disclosure can be better appreciated with reference to FIGS. 15-17. For purposes of simplicity of explanation, example methods disclosed herein are presented and described as a series of acts. The example methods are not limited by the order of the acts, as some acts may occur in different orders and/or concurrently with other acts from that shown and described herein. In some cases, one or more example methods disclosed herein can alternatively be represented as a series of interrelated states or events, such as in a state diagram depicting a state machine. In addition, or in other cases, interaction diagram(s) (or process flow(s)) may represent methods in accordance with aspects of this disclosure when different entities enact different portions of the methodologies. It is noted that not all illustrated acts may be required to implement a described example method in accordance with this disclosure. It is also noted that two or more of the disclosed example methods can be implemented in combination with each other, to accomplish one or more functionalities described herein.

Methods disclosed herein can be stored on an article of manufacture in order to permit or otherwise facilitate transporting and transferring such methodologies to headphones and other types of hearing aid devices, and computing devices (such as handheld, portable, or tethered computing devices) or other types of information processing apparatuses for execution, and thus implementation, by one or more processors, individually or in combination, or for storage in a memory device or another type of computer-readable storage device. In one example, one or more processors that enact a method or combination of methods described herein can be utilized to execute program code retained in a memory device, or any processor-readable or machine-readable storage device or non-transitory storage media, in order to implement method(s) described herein. The program code, when configured in processor-executable form and executed by the one or more processors, causes the implementation or performance of the various acts in the method(s) described herein. The program code thus provides a processor-executable or machine-executable framework to enact the method(s) described herein. Accordingly, in some cases, each block of the flowchart illustrations and/or combinations of blocks in the flowchart illustrations can be implemented in response to execution of the program code.

FIG. 15 is a flowchart of an example of a method for performing a hearing test in accordance with one or more aspects of this disclosure. Similar to other methods for performing a hearing test that are disclosed herein, a control system can implement the example method 1500. The control system is in some cases the control system 910 (FIGS. 9, 12, 13).

At block 1510, the control system can obtain contextual information associated with a subject and/or an environment of headphones configured to apply the hearing test. As is described herein, the contextual information includes at least one of physiological data of the subject, acoustic characteristics of the environment, or anatomical characteristics of the subject. Because the subject wears the headphones during the hearing test, the environment of the headphones forms or is part of the environment of the subject.

In some cases, as is described herein, obtaining the contextual information includes causing a context module to detect an amount of sound leakage passing through the headphones. The context module can be integrated into or co-located with a headphone device (such as an earphone or earbud) of the headphones. The context module includes an ANC module and at least one microphone that can detect the amount of sound leakage passing through the headphones, in accordance with aspects described herein. In one example, the context module is or includes the leakage detection module 1310 (FIG. 13) described herein, and the at least one microphone includes one or multiple ones of the microphone(s) 1204.

In addition, or in other cases, as is described herein, obtaining the contextual information comprises causing a second context module to detect a vocalization produced by the subject during the hearing test. The second context module can be integrated into or co-located with a headphone device (such as an earphone or earbud) of the headphones. In one example, the second context module is or includes the OVD module 1320 (FIG. 13).

Further, or in yet other cases, as is described herein, obtaining the contextual information includes determining tinnitus frequency information corresponding to the subject. The tinnitus frequency information can be determined in numerous ways. In one example, the tinnitus frequency information is determined by measuring one or more tinnitus frequencies of the subject. In addition, or in another example, the tinnitus information is determined by receiving input information indicative of tinnitus frequencies of the subject.

Furthermore, or in still other cases, as is described herein, obtaining the contextual information includes causing a third context module to measure at least one physiological attribute of the subject including at least one of heart rate, temperature, or a value SpO2. The third context module includes one or more VSM sensor devices integrated into or coupled with the headphones. In one example, the third context module is or includes the VSM module 1340 (FIG. 13) described herein.

At block 1520, the control system can cause one or more test signals to be conditioned based on the contextual information, resulting in one or more conditioned test signals. To that end, in some cases, the control system can cause one or more functional elements of the headphones to condition the one or more test signals. The one or more functional elements of the headphones include, in some configurations, a tone generator, a compensation element, or one or more context modules in accordance with aspects of this disclosure. In one example, the functional elements include the tone generator 904, the compensation element 902, and the context module(s) 1230 (FIGS. 12, 13). Conditioning the one or more test signals can include adjusting a frequency of the one or more test signals, adjusting an amplitude of the one or more test signals, and/or configuring a spatial rendering of the one or more test signals.

In cases where sound leakage passing through the headphones is detected as part of obtaining contextual information, conditioning the one or more test signals based on the contextual information can include compensating, by an ANC module of the context module, for the detected sound leakage by continuously monitoring a feedback microphone response inside an ear canal of the subject and adjusting noise cancellation filters to maintain a consistent ANC response.

In cases where tinnitus frequency information corresponding to the subject is determined, conditioning the one or more test signals based on the contextual information includes adjusting the one or more test signals to avoid having respective frequencies overlapping the tinnitus frequency or tinnitus frequencies defined by the tinnitus frequency information. Adjusting the one or more test signals in such a fashion includes, for example, shifting the one or more test signals to frequencies outside a frequency range including the tinnitus frequency or tinnitus frequencies of the subject. In addition, or in some configurations, conditioning the one or more test signals based on the contextual information includes increasing an amplitude of the one or more test signals at frequencies within the tinnitus frequency range to overcome masking by the tinnitus.

Configuring the spatial rendering of the one or more test signals includes, for example, pre-filtering the one or more test signals using an HRTF to spatially render the one or more test signals as perceived by the subject as originating from one or more directions in three-dimensional space. As is described herein, the HRTF can correspond to the subject or can be a generic HRTF. In cases the HRTF corresponds to the subject undergoing the hearing test, the control system can cause a computing device (e.g., a mobile device, such as the mobile device 1202 (FIG. 12)) that hosts the control system or is coupled with the control system to capture anatomical characteristics of the subject including at least one of a geometry of a head, geometry of a face, or morphology of ears of the subject. The control system can further cause the computing device to generate, based on the captured anatomical characteristics, the HRTF corresponding to the subject. The HRTF can be generated in accordance with aspects described herein. In cases the HRTF is generic, the HRTF corresponds to or a model of subject (e.g., a dummy or mannequin representing an adult, an adolescent, or a child, depending on subject).

At block 1530, the control system can receive respective indications of SPL of the one or more conditioned test signals. Each one of the respective indications may be a digital signal defining the SPL of a respective conditioned test signal. As is described herein, SPL of a conditioned test signal can be measured by a headphone device (such as an earphone or an earbud) of the headphones. To that end, a tone generator integrated into the headphone device can cause a conditioned signal to be emitted by a speaker unit integrated into or co-located within the headphone device. Further, a microphone integrated into or co-located within the headphone device can detect the conditioned test signal emitted by the speaker. Furthermore, an SPL measurement element integrated into or co-located with the headphone device and coupled with the microphone, can determine the SPL of the conditioned test signal. The SPL measurement can supply, e.g., send or otherwise make available, an indication of the SPL of the conditioned test signal. In one example, the tone generator is the tone generator 904 (FIG. 12), the speaker unit is the speaker device 1206 (or speaker 1206; FIG. 12), the microphone is one of the microphone(s) 1204 (FIG. 12), and the SPL measurement element is the SPL measurement element 1205 (FIG. 12).

At block 1540, the control system can receive an indication of whether the subject heard at least one of the one or more conditioned test signals.

At block 1550, the control system can generate a hearing profile based on the SPL of the one or more conditioned test signals and the indication of whether the subject heard the one or more conditioned test signals. The hearing profile that is generated corresponds to the subject undergoing the hearing test and contextual information.

At block 1560, the control system can retain the hearing profile, including the contextual information, in a non-volatile memory device for use in hearing compensation of subsequent sounds emitted by a speaker of the headphones. The hearing compensation is based on both the hearing profile and the contextual information therein. In one example, the non-volatile memory device is part of the memory 1210 (FIG. 12).

In cases where the headphones include a context module that can detect a vocalization of a subject undergoing the hearing text, the contextual information indicative of such a vocalization can be used to control aspects of the hearing test. More specifically, in those cases, the example method 1500 can include rejecting or accepting a test measurement performed during a detected vocalization. In an instance in which the test measurement is rejected, the example method 1500 can include repeating the test measurement.

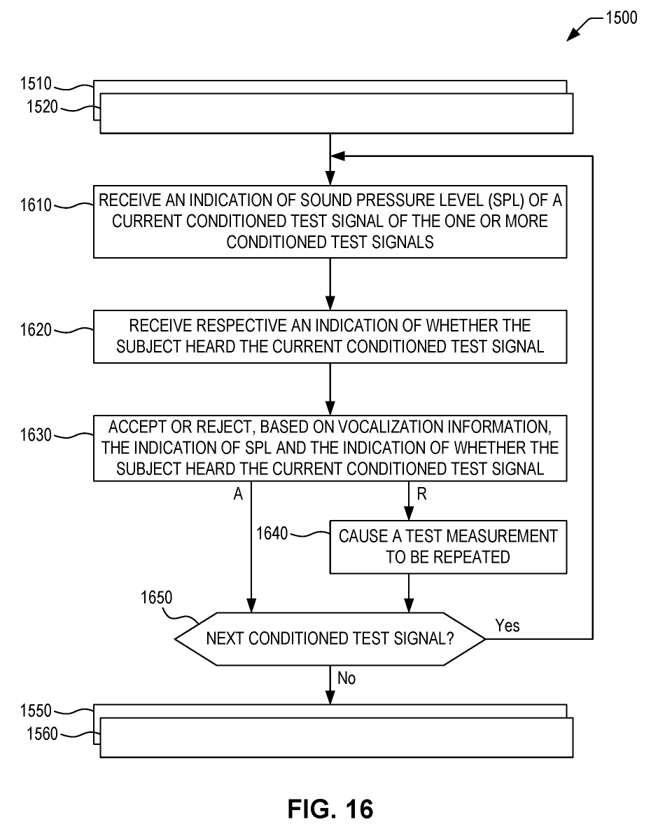

For purposes of illustration of vocalization aspects, FIG. 16 is a flowchart the example method 1500 in configurations where a vocalization can be detected. Besides implementing block 1510 and block 1520, at block 1610 the control system can receive an indication of SPL of a current conditioned test signal of the one or more conditioned test signals. The indication may be a digital signal defining the SPL of the test signal. As is described herein, SPL of a conditioned test signal can be measured by a headphone device (such as an earphone or an earbud) of the headphones.

At block 1620, the control system can receive an indication of whether the subject heard the current conditioned test signals. Because vocalization aspects are included, the control system can receive an aural response from the subject indicating whether the subject heard the current conditioned test signal. A context module configured to detect vocalizations can provide a voice user interface that receives and processes the aural response. The voice user interface also supplies the aural response and/or a digital signal representative of the aural response to the control system. In one example, the context module is or includes the OVD module 1320 (FIG. 13).

At block 1630, the control system can either accept or reject, based on vocalization information, the indication of SPL and the indication of whether the subject heard the current conditioned test signal. The vocalization information is part of the contextual information obtained at block 1510 and indicates that a vocalization is present or absent during the hearing test. The context module that provides the aural response at block 1620 can detect the vocalization. In the absence of a vocalization (“A” branch in FIG. 16) the flow continues to block 1650. In the presence of a vocalization (“R” branch) the flow continues to block 1640 where the control system can cause a test measurement corresponding to the current conditioned test signal to be repeated. The flow then continues to block 1650.

At block 1650, the control system can determine if a next conditioned test signal is to be used in a next test measurement. An affirmative determination causes the flow of the example method 1500 to return to and continue from block 1610. A negative determination is indicative of completing test measurements corresponding to the one or more conditioned test measurements. The negative determination causes the flow of the example method 1500 to continue to block 1550 and block 1560.

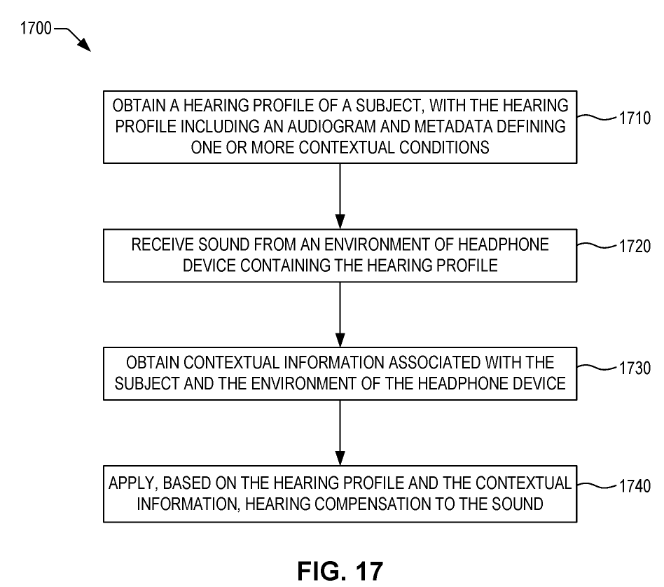

FIG. 17 is a flowchart of an example of a method for compensating for hearing loss in accordance with one or more aspects of this disclosure. A headphone device, such as an earbud or an earphone, in accordance with aspects described herein can implement the example method 1700 in its entirety or partially. The headphone device can serve as a hearing aid device or a lifestyle hearing device, or both. In some cases, the headphone device is part of the headphones included in the example system 1200 (FIGS. 12, 13, and 14) described herein. In one example where the headphone device is or includes the hearing device 1405 (FIG. 14), the headphone device can implement the example method 1500 by executing at least one of the modules present within the memory 1430.

At block 1710, the headphone device can obtain a hearing profile of a subject. The hearing profile includes at least one audiogram and respective metadata defining one or more contextual conditions. That is, a first audiogram of the at least one audiogram includes first metadata defining contextual condition(s). In cases multiple audiograms are configured, a second audiogram includes second metadata defining other contextual condition(s). Obtaining the hearing profile includes, in some cases, receiving the hearing profile from a control system communicatively coupled with the headphone device. In addition, in those cases, the obtaining also includes retaining the hearing profile in a non-volatile memory device integrated into the headphone device. The non-volatile memory device is part of the memory 1240 in one example.

At block 1720, the headphone device can receive sound from an environment of the headphone device. One or more microphones integrated into the headphone device or functionally coupled therewith can receive the sound. In some cases, a subject wears the headphone device, and the environment of the headphone device thus forms or is part of the environment of the subject. The one or more microphones are part of or can include the microphone(s) 1204 (FIG. 12) in one example.

At block 1730, the headphone device can obtain contextual information corresponding to the subject wearing the headphone device and the environment of the headphone device. As is described herein, the contextual information can be obtained by one or multiple context modules 1230 (FIGS. 12, 13, and 14) integrated into or coupled with the headphone device. The contextual information that is obtained includes at least one of physiological data of the subject, acoustic characteristics of the environment, or a direction of sound.

At block 1740, the headphone device can apply, based on the hearing profile for the subject and the contextual information, hearing compensation to the sound. To that end, in some cases, the headphone device includes a compensation module (such as the compensation element 902 (FIGS. 12, 13, and 14)). Applying the hearing compensation in such a fashion includes at least one of executing a prescription procedure for providing wide dynamic range compression or implementing sound personalization tuning. Executing the prescription procedure includes evaluating a defined fitting formula. In some configurations, the compensation module includes a processor configured to execute the compensation module, and in response, execute the prescription procedure and/or implement the sound personalization tuning. In other configurations, the compensation module can be implemented as processor-accessible instructions. In such configurations, the hearing device includes a processor configured to execute, individually or in combination, the compensation module to execute the prescription procedure and/or implement the sound personalization tuning.

The foregoing outlines features of one or more embodiments of the subject matter disclosed herein. These embodiments are provided to enable a person having ordinary skill in the art (PHOSITA) to better understand various aspects of the present disclosure. Certain well-understood terms, as well as underlying technologies and/or standards may be referenced without being described in detail. It is anticipated that the PHOSITA will possess or have access to background knowledge or information in those technologies and standards sufficient to practice the teachings of the present disclosure.

The PHOSITA will appreciate that they may readily use the present disclosure as a basis for designing or modifying other processes, structures, or variations for carrying out the same purposes and/or achieving the same advantages of the embodiments introduced herein. The PHOSITA will also recognize that such equivalent constructions do not depart from the spirit and scope of the present disclosure, and that they may make various changes, substitutions, and alterations herein without departing from the spirit and scope of the present disclosure.

Note that the activities discussed above with reference to the FIGURES are applicable to any integrated circuit that involves signal processing (for example, gesture signal processing, video signal processing, audio signal processing, analog-to-digital conversion, digital-to-analog conversion), particularly those that can execute specialized software programs or algorithms, some of which may be associated with processing digitized real-time data. Certain embodiments can relate to multi-DSP, multi-ASIC, or multi-SoC signal processing, floating point processing, signal/control processing, fixed-function processing, microcontroller applications, etc. In certain contexts, the features discussed herein can be applicable to medical systems, scientific instrumentation, wireless and wired communications, radar, industrial process control, audio and video equipment, current sensing, instrumentation (which can be highly precise), and other digital-processing-based systems. Moreover, certain embodiments discussed above can be provisioned in digital signal processing technologies for medical imaging, patient monitoring, medical instrumentation, and home healthcare. This could include, for example, pulmonary monitors, accelerometers, heart rate monitors, or pacemakers, along with peripherals therefor. Other applications can involve automotive technologies for safety systems (e.g., stability control systems, driver assistance systems, braking systems, infotainment and interior applications of any kind). Furthermore, powertrain systems (for example, in hybrid and electric vehicles) can use high-precision data conversion, rendering, and display products in battery monitoring, control systems, reporting controls, maintenance activities, and others. In yet other example scenarios, the teachings of the present disclosure can be applicable in the industrial markets that include process control systems that help drive productivity, energy efficiency, and reliability. In consumer applications, the teachings of the signal processing circuits discussed above can be used for image processing, auto focus, and image stabilization (e.g., for digital still cameras, camcorders, etc.). Other consumer applications can include audio and video processors for home theater systems, DVD recorders, and high-definition televisions. Yet other consumer applications can involve advanced touch screen controllers (e.g., for any type of portable media device). Hence, such technologies could readily be part of smartphones, tablets, security systems, PCs, gaming technologies, virtual reality, simulation training, etc.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

The particular embodiments of the present disclosure may readily include a system on chip (SoC) central processing unit (CPU) package. An SoC represents an integrated circuit (IC) that integrates components of a computer or other electronic system into a single chip. It may contain digital, analog, mixed-signal, and radio frequency functions: all of which may be provided on a single chip substrate. Other embodiments may include a multi-chip-module (MCM), with a plurality of chips located within a single electronic package and configured to interact closely with each other through the electronic package. Any module, function, or block element of an ASIC or SoC can be provided, where appropriate, in a reusable “black box” intellectual property (IP) block, which can be distributed separately without disclosing the logical details of the IP block. In various other embodiments, the digital signal processing functionalities may be implemented in one or more silicon cores in application-specific integrated circuits (ASICs), field-programmable gate arrays (FPGAs), and other semiconductor chips.

In some cases, the teachings of the present disclosure may be encoded into one or more tangible, non-transitory computer-readable mediums having stored thereon executable instructions that, when executed, instruct a programmable device (such as a processor or DSP) to perform the methods or functions disclosed herein. In cases where the teachings herein are embodied at least partly in a hardware device (such as an ASIC, IP block, or SoC), a non-transitory medium could include a hardware device hardware-programmed with logic to perform the methods or functions disclosed herein. The teachings could also be practiced in the form of Register Transfer Level (RTL) or other hardware description language such as VHDL or Verilog, which can be used to program a fabrication process to produce the hardware elements disclosed.

In example implementations, at least some portions of the processing activities outlined herein may also be implemented in software. In some embodiments, one or more of these features may be implemented in hardware provided external to the elements of the disclosed figures, or consolidated in any appropriate manner to achieve the intended functionality. The various components may include software (or reciprocating software) that can coordinate in order to achieve the operations as outlined herein. In still other embodiments, these elements may include any suitable algorithms, hardware, software, components, modules, interfaces, or objects that facilitate the operations thereof.

Additionally, some of the components associated with described microprocessors may be removed, or otherwise consolidated. In a general sense, the arrangements depicted in the figures may be more logical in their representations, whereas a physical architecture may include various permutations, combinations, and/or hybrids of these elements. It is imperative to note that countless possible design configurations can be used to achieve the operational objectives outlined herein. Accordingly, the associated infrastructure has a myriad of substitute arrangements, design choices, device possibilities, hardware configurations, software implementations, equipment options, etc.

Any suitably-configured processor component can execute any type of instructions associated with the data to achieve the operations detailed herein. Any processor disclosed herein could transform an element or an article (for example, data) from one state or thing to another state or thing. In another example, some activities outlined herein may be implemented with fixed logic or programmable logic (for example, software and/or computer instructions executed by a processor) and the elements identified herein could be some type of a programmable processor, programmable digital logic (for example, an FPGA, an erasable programmable read only memory (EPROM), an electrically erasable programmable read only memory (EEPROM)), an ASIC that includes digital logic, software, code, electronic instructions, flash memory, optical disks, CD-ROMs, DVD ROMs, magnetic or optical cards, other types of machine-readable mediums suitable for storing electronic instructions, or any suitable combination thereof. In operation, processors may store information in any suitable type of non-transitory storage medium (for example, random access memory (RAM), read only memory (ROM), FPGA, EPROM, electrically erasable programmable ROM (EEPROM), etc.), software, hardware, or in any other suitable component, device, element, or object where appropriate and based on particular needs. Further, the information being tracked, sent, received, or stored in a processor could be provided in any database, register, table, cache, queue, control list, or storage structure, based on particular needs and implementations, all of which could be referenced in any suitable timeframe. Any of the memory items discussed herein should be construed as being encompassed within the broad term ‘memory.’ Similarly, any of the potential processing elements, modules, and machines described herein should be construed as being encompassed within the broad term ‘microprocessor’ or ‘processor.’ Furthermore, in various embodiments, the processors, memories, network cards, buses, storage devices, related peripherals, and other hardware elements described herein may be realized by a processor, memory, and other related devices configured by software or firmware to emulate or virtualize the functions of those hardware elements.

Computer program logic implementing all or part of the functionality described herein is embodied in various forms, including, but in no way limited to, a source code form, a computer executable form, a hardware description form, and various intermediate forms (for example, mask works, or forms generated by an assembler, compiler, linker, or locator). In an example, source code includes a series of computer program instructions implemented in various programming languages, such as an object code, an assembly language, or a high-level language such as OpenCL, RTL, Verilog, VHDL, Fortran, C, C++, JAVA, or HTML for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

In the discussions of the embodiments above, the capacitors, buffers, graphics elements, interconnect boards, clocks, DDRs, camera sensors, converters, inductors, resistors, amplifiers, switches, digital core, transistors, and/or other components can readily be replaced, substituted, or otherwise modified in order to accommodate particular circuitry needs. Moreover, it should be noted that the use of complementary electronic devices, hardware, non-transitory software, etc. offer an equally viable option for implementing the teachings of the present disclosure.

In one example embodiment, any number of electrical circuits of the FIGURES may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), memory elements, etc. can be suitably coupled to the board based on particular configuration needs, processing demands, computer designs, etc. Other components such as external storage, additional sensors, controllers for audio/video display, and peripheral devices may be attached to the board as plug-in cards, via cables, or integrated into the board itself. In another example embodiment, the electrical circuits of the FIGURES may be implemented as standalone modules (e.g., a device with associated components and circuitry configured to perform a specific application or function) or implemented as plug-in modules into application-specific hardware of electronic devices.

Note that with the numerous examples provided herein, interaction may be described in terms of two, three, four, or more electrical components. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this disclosure. In certain cases, it may be easier to describe one or more of the functionalities of a given set of flows by only referencing a limited number of electrical elements. It should be appreciated that the electrical circuits of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

### Example Implementations

The following examples are provided by way of illustration.

Example 1 may include a system to be located within headphones for performing hearing testing, comprising a tone generator to receive an indication of a tone to be emitted from the headphones, and cause the tone to be emitted by a speaker within the headphones, and a sound pressure level (SPL) measurement element to receive an indication of the tone detected by a microphone within the headphones; and determine an SPL of the tone detected by the microphone, wherein the SPL is to be used to verify that the SPL is equal to an intended SPL for the tone.

Example 2 may include the system of example 1, wherein SPL is to be used to calibrate the tone generator for subsequent tones to be emitted by the speaker.

Example 3 may include the system of example 1, further comprising an active noise cancellation (ANC) element coupled to an output of the tone generator and an input of the SPL measurement element, the ANC element to determine an indication of sounds detected by the microphone, the sounds being separate from the tone, and apply ANC with the speaker based on the indication of the sounds to noise cancel the sounds.

Example 4 may include the system of example 1, further comprising a compensation element coupled to an output of the tone generator, the compensation element to apply compensation to signal provided to the compensation element.

Example 5 may include the system of example 1, further comprising a control system to provide the indication of the tone to be emitted to the tone generator, and utilize the SPL of the tone to calibrate the tone generator for subsequent tones to be emitted by the speaker.

Example 6 may include the system of example 5, wherein the control system is further to generate an audiogram based on the hearing testing, and cause compensation to be applied to sounds to be emitted from the speaker.

Example 7 may include the system of example 5, wherein the control system is further to detect an indication that a hearing test is to be performed, and wherein the control system is to provide the indication of the tone in response to detection of the indication that the hearing test is to be performed.

Example 8 may include the system of example 1, further comprising a bias removal element to cause an unbiased approach to be utilized for the hearing testing.

Example 9 may include the system of example 1, wherein the system is implemented within a processor located within the headphones.

Example 10 may include headphones, comprising a speaker to emit tones, a tone generator coupled to the speaker to cause the speaker to emit a first tone, a microphone to detect the first tone emitted by the speaker, and a sound pressure level (SPL) measurement element coupled to the microphone to determine an SPL of the first tone emitted by the speaker based on detection of the first tone by the microphone, wherein the determined SPL is to be utilized to determine whether to adjust SPLs of the tones to be emitted by the speaker.

Example 11 may include the headphones of example 10, further comprising a control system coupled to the tone generator and the SPL measurement element, the control system to compare the determined SPL of the first tone with an intended SPL for the first tone, and control the SPLs of the tones based on a results of the comparison of the determined SPL with the intended SPL.

Example 12 may include the headphones of example 11, wherein the speaker is further to emit sounds provided by a remote device, wherein the control system is further to control SPLs of the sounds based on the results of the comparison of the determined SPL with the intended SPL.

Example 13 may include the headphones of example 11, wherein the control system is further to detect an indication that a hearing test is to be performed, and provide an indication to the tone generator to cause the speaker to emit the first tone in response to detection of the indication that the hearing test is to be performed.

Example 14 may include the headphones of example 11, further comprising a bias removal element coupled to an input of the control system, wherein the bias removal element is to implement an unbiased testing approach for hearing testing to be performed by the headphones.

Example 15 may include the headphones of example 10, further comprising an active noise cancellation (ANC) element coupled to the speaker and the microphone, the ANC element to identify environmental noises detected by the microphone, and apply ANC to the tones to be emitted by the speaker, the ANC based on the identified environmental noises.

Example 16 may include the headphones of example 15, further comprising a digital-to-analog converter (DAC) coupled between the tone generator and the speaker, wherein the ANC element is coupled to the speaker between the tone generator and the DAC, and an analog-to-digital converter (ADC) coupled between the SPL measurement element and microphone, wherein the ANC element is coupled to the microphone between the SPL measurement element and the ADC.

Example 17 may include the headphones of example 10, wherein the speaker, the tone generator, the microphone, and the SPL measurement element co-located within an earphone or an earbud of the microphone.

Example 18 may include a method of performing a hearing test by headphones, comprising causing, by a tone generator located within the headphones, a tone to be emitted by a speaker of the headphones, detecting, by a microphone located within the headphones, the tone emitted by the speaker, determining, by a sound pressure level (SPL) located within the headphones, an SPL of the tone emitted by the speaker, comparing the determined SPL of the tone with an intended SPL for the tone, and determining whether to adjust SPLs of subsequent tones to be emitted by the speaker based on the comparison of the determined SPL of the tone with the intended SPL of the tone.

Example 19 may include the method of example 18, further comprising identifying, by an active noise cancellation (ANC) element within the headphones, environmental noise detected by the microphone, and applying, by the ANC element, ANC to the subsequent tones to be emitted by the speaker.

Example 20 may include the method of example 18, further comprising applying, by a compensation element within the headphones, compensation to sounds to be emitted by the speaker, the compensation based on the comparison of the determined SPL of the tone with the intended SPL of the tone.

Example 21. A method for performing a hearing test, comprising: obtaining, by a control system, contextual information associated with a subject and an environment of headphones configured to apply the hearing test, the contextual information including at least one of physiological data of the subject, acoustic characteristics of the environment, or anatomical characteristics of the subject; causing, by the control system, one or more test signals to be conditioned based on the contextual information, resulting in one or more conditioned test signals; receiving, by the control system, respective indications of sound pressure level (SPL) of the one or more conditioned test signals; receiving, by the control system, respective indications of whether the subject heard the one or more conditioned test signals; generating, by the control system, a hearing profile of the subject based on the respective indications of SPL of the one or more conditioned test signals and the respective indications of whether the subject heard the one or more conditioned test signals; and retaining, by the control system, the hearing profile and the contextual information in a non-volatile memory device for use in hearing compensation of subsequent sounds emitted by a speaker device of the headphones, with the hearing compensation being based on the hearing profile and the contextual information.

Example 22. The method of Example 21, wherein the obtaining comprises causing detection of an amount of sound leakage passing through the headphones.

Example 23. The method of any of the preceding Examples, wherein the conditioning comprises compensating for the detected sound leakage by continuously monitoring a feedback microphone response and adjusting noise cancellation filters to maintain a consistent active noise cancellation (ANC) response within a headphone device of the headphones.

Example 24. The method of any of the preceding Examples, wherein the obtaining comprises causing detection of a vocalization during the hearing test.

Example 25. The method of any of the preceding Examples, wherein the method further comprises, rejecting, by the control system, a test measurement performed during the detected vocalization; and causing, by the control system, the test measurement to be repeated.

Example 26. The method of any of the preceding Examples, wherein the obtaining comprises determining tinnitus frequency information corresponding to the subject, with the tinnitus frequency information being indicative of one or more tinnitus frequencies.

Example 27. The method of any of the preceding Examples, wherein the conditioning comprises at least one of: adjusting the one or more test signals to avoid having respective frequencies overlapping the one or more tinnitus frequencies, or increasing an amplitude of the one or more test signals at frequencies within a frequency range that includes the one or more tinnitus frequencies.

Example 28. The method of any of the preceding Examples, wherein the obtaining comprises causing measurement of at least one physiological attribute of the subject including at least one of heart rate, temperature, or a value of blood oxygen saturation (SpO2).

Example 29. The method of any of the preceding Examples, wherein the conditioning comprises at least one of: adjusting at least one of a frequency of the one or more test signals, adjusting an amplitude of the one or more test signals, or configuring a spatial rendering of the one or more test signals.

Example 30. The method of any of the preceding Examples, wherein the configuring the spatial rendering of the one or more test signals comprises pre-filtering the one or more test signals using a head-related transfer function (HRTF) to spatially render the one or more test signals as perceived as originating from one or more directions in three-dimensional space.

Example 31. The method of any of the preceding Examples, wherein the HRTF corresponds to the subject, the method further comprising, causing, by the control system, a computing device to capture anatomical characteristics of the subject including at least one of a geometry of a head, geometry of a face, or morphology of ears of the subject; and causing, by the control system, the computing device, to generate the HRTF based on the captured anatomical characteristics.

Example 32. The method of any of the preceding Examples, with the one or more directions including multiple directions having respective angles distributed spherically around the subject.

Example 33. A method of compensating for hearing loss, comprising: configuring, within a headphone, a hearing profile of a subject, with the hearing profile including an audiogram and metadata defining one or more contextual conditions; receiving, by one or more microphones integrated into the headphone, sound from an environment of the subject; obtaining, by the headphone, contextual information associated with the subject and the environment of the subject, the contextual information including at least one of physiological data of the subject, acoustic characteristics of the environment, or a direction of sound; and applying, based on the hearing profile and the contextual information, hearing compensation to the sound.

Example 34. The method of Example 33, wherein the applying the hearing compensation comprises: dynamically updating, by a compensation element within the headphone, the hearing compensation based on at least one physiological attribute defined by the physiological data, wherein the dynamically updating is performed using a machine learning module that correlates the at least one physiological attribute with hearing performance of the subject.

Example 35. The method of any of the preceding Examples, wherein the applying the hearing compensation comprises at least one of executing a prescription procedure for providing wide dynamic range compression or implementing sound personalization tuning.

Example 36. The method of any of the preceding Examples, wherein executing the prescription procedure comprises evaluating a defined fitting formula.

Example 37. A system, comprising: one or more memory devices; one or more context modules; and a control system communicatively coupled with the one or more context modules and the one or more memory devices, the control system configured to: obtain, using at least one context module of the one or more context modules, contextual information associated with a subject and an environment of headphones configured to apply a hearing test, with the contextual information including at least one of physiological data of the subject, acoustic characteristics of the environment, or anatomical characteristics of the subject; cause one or more test signals to be conditioned based on the contextual information, resulting in one or more conditioned test signals; receive respective first indications of SPL of the one or more conditioned test signals; receive respective second indications of whether the subject heard the one or more conditioned test signals; generate a hearing profile of the subject based on the respective first indications and the respective second indications; and retain the hearing profile and the contextual information in a non-volatile memory of the one or more memory devices for use in hearing compensation of subsequent sounds emitted by a speaker device, with the hearing compensation being based on the hearing profile and the contextual information.

Example 38. The system of Example 37, wherein the at least one context module comprises a leakage detection module having an adaptive active noise cancellation (ANC) module, and wherein to obtain the contextual information, the control system is further configured to cause the leakage detection module to detect an amount of sound leakage passing through the headphones.

Example 39. The system of any of the preceding Examples, wherein the leakage detection module further has a feedback microphone, and wherein conditioning the one or more test signals comprises compensating for detected sound leakage by continuously monitoring a response of the feedback microphone and adjusting noise cancellation filters to maintain a consistent ANC response.

Example 40. The system of any of the preceding Examples, wherein the at least one context module comprises an own-voice detection module configured to detect a vocalization during the hearing test, wherein to obtain the contextual information, the control system is further configured to cause the own-voice detection module to determine presence or absence of the vocalization during the hearing test.

Example 41. The system of any of the preceding Examples, wherein the control system is further configured to, reject a test measurement performed during presence of the vocalization; and cause the test measurement to be repeated.

Example 42. The system of any of the preceding Examples, wherein to obtain the contextual information, the control system is further configured to determine tinnitus frequency information corresponding to the subject, with the tinnitus frequency information being indicative of one or more tinnitus frequencies.

Example 43. The system of any of the preceding Examples, wherein to condition the one or more test signals, the control system is further configured to, adjust the one or more test signals to avoid having respective frequencies overlapping the one or more tinnitus frequencies, or increase an amplitude of the one or more test signals at frequencies within a frequency range that includes the one or more tinnitus frequencies.

Example 44. The system of any of the preceding Examples, wherein the at least one context module comprises a vital sign monitoring (VSM) module configured to measure at least one physiological attribute including at least one of heart rate, temperature, or blood oxygen saturation (SpO2), wherein to obtain the contextual information, the control system is further configured to cause the VSM module to measure one or more physiological attributes.

Example 45. The system of any of the preceding Examples, wherein conditioning the one or more test signals comprises configuring a spatial rendering of the one or more test signals, with the configuring including pre-filtering the one or more test signals using a head-related transfer function (HRTF) to spatially render the one or more test signals as perceived as originating from one or more directions in three-dimensional space.

Example 46. A headphone device for hearing loss compensation, comprising: one or more microphones integrated into the headphone device and configured to receive sound from an environment of the headphone device; a memory device having stored thereon a hearing profile of a subject, with the hearing profile including an audiogram and metadata defining one or more contextual conditions; one or more context modules configured to obtain contextual information associated with the subject and the environment of the headphone device, the contextual information including at least one of (i) physiological data of the subject or (ii) acoustic characteristics of the environment; and a compensation module configured to apply, based on the hearing profile and the contextual information, hearing compensation to the sound.

Example 47. The headphone device of Example 46, wherein the compensation module is further configured to update the hearing compensation based on at least one physiological attribute defined by the physiological data.

Example 48. The headphone device of any of the preceding Examples, further comprising a machine-learning module configured to correlate the at least one physiological attribute with hearing performance of the subject.

Example 49. The headphone device of any of the preceding Examples, wherein applying the hearing compensation to the sound comprises at least one of implementing a prescription procedure for providing wide dynamic range compression or implementing sound personalization tuning.

Example 50. The headphone device of any of the preceding Examples, wherein the implementing the prescription procedure comprises evaluating a defined fitting formula.

Various aspects of the disclosure may take the form of an entirely or partially hardware aspect, an entirely or partially software aspect, or a combination of software and hardware. Furthermore, as described herein, various aspects of the disclosure (e.g., systems and methods) may take the form of a computer program product comprising a computer-readable non-transitory storage medium having computer-accessible instructions (e.g., computer-readable and/or computer-executable instructions) such as computer software, encoded or otherwise embodied in such storage medium. Those instructions can be read or otherwise accessed and executed by one or more processors to perform or permit the performance of the operations described herein. The instructions can be provided in any suitable form, such as source code, compiled code, interpreted code, executable code, static code, dynamic code, assembler code, combinations of the foregoing, and the like. Any suitable computer-readable non-transitory storage medium may be utilized to form the computer program product. For instance, the computer-readable medium may include any tangible non-transitory medium for storing information in a form readable or otherwise accessible by one or more computers or processor(s) functionally coupled thereto. Non-transitory storage media can include read-only memory (ROM); random access memory (RAM); magnetic disk storage media; optical storage media; flash memory, and so forth.

Aspects of this disclosure are described herein with reference to block diagrams and flowchart illustrations of methods, systems, apparatuses and computer program products. It can be understood that each block of the block diagrams and flowchart illustrations, and combinations of blocks in the block diagrams and flowchart illustrations, respectively, can be implemented by computer-accessible instructions. In certain implementations, the computer-accessible instructions may be loaded or otherwise incorporated into a general purpose computer, a special purpose computer, or another programmable information processing apparatus to produce a particular machine, such that the operations or functions specified in the flowchart block or blocks can be implemented in response to execution at the computer or processing apparatus.

Unless otherwise expressly stated, it is in no way intended that any protocol, procedure, process, or method set forth herein be construed as requiring that its acts or steps be performed in a specific order. Accordingly, where a process or method claim does not actually recite an order to be followed by its acts or steps or it is not otherwise specifically recited in the claims or descriptions of the subject disclosure that the steps are to be limited to a specific order, it is in no way intended that an order be inferred, in any respect. This holds for any possible non-express basis for interpretation, including: matters of logic with respect to the arrangement of steps or operational flow; plain meaning derived from grammatical organization or punctuation; the number or type of aspects described in the specification or annexed drawings; or the like.

As used in this disclosure, including the annexed drawings, the terms “component,” “module,” “system,” and the like are intended to refer to a computing/processing-related entity or an entity related to an apparatus or a combination of apparatuses each having one or more specific functionalities. The entity can be either hardware, a combination of hardware and software, software, or software in execution. One or more of such entities are also referred to as “functional elements” or “elements.” As an example, a component can be a process running on a processor, a processor, an object, an executable, a thread of execution, a program, and/or a computer. For example, both an application running on a server or network controller, and the server or network controller can be a component. One or more components can reside within a process and/or thread of execution and a component can be localized on one computer and/or distributed between two or more computers. Also, these components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which parts can be controlled or otherwise operated by program code executed by a processor. As yet another example, a component can be an apparatus that provides specific functionality through electronic components without mechanical parts, the electronic components can include a processor to execute program code that provides, at least partially, the functionality of the electronic components. As still another example, interface(s) can include I/O components or Application Programming Interface (API) components. While the foregoing examples are directed to aspects of a component, the exemplified aspects or features also apply to a system, module, and similar.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in this specification and annexed drawings should be construed to mean “one or more” unless specified otherwise or clear from context to be directed to a singular form.

In addition, the terms “example” and “such as” are utilized herein to mean serving as an instance or illustration. Any aspect or design described herein as an “example” or referred to in connection with a “such as” clause is not necessarily to be construed as preferred or advantageous over other aspects or designs described herein. Rather, use of the terms “example” or “such as” is intended to present concepts in a concrete fashion. The terms “first,” “second,” “third,” and so forth, as used in the claims and description, unless otherwise clear by context, is for clarity only and doesn't necessarily indicate or imply any order in time or space.

The term “processor,” as utilized in this disclosure, can refer to any computing processing unit or device comprising processing circuitry that can operate on data and/or signaling. A computing processing unit or device can include, for example, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can include an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. In some cases, processors can exploit nano-scale architectures, such as molecular and quantum-dot based transistors, switches and gates, in order to optimize space usage or enhance performance of user equipment. A processor may also be implemented as a combination of computing processing units.

In addition, terms such as “store,” “data store,” data storage,” “database,” and substantially any other information storage component relevant to operation and functionality of a component, refer to “memory components,” or entities embodied in a “memory” or components comprising the memory. It will be appreciated that the memory components described herein can be either volatile memory or nonvolatile memory, or can include both volatile and nonvolatile memory. Moreover, a memory component can be removable or affixed to a functional element (e.g., device, server).

Simply as an illustration, nonvolatile memory can include read only memory (ROM), programmable ROM (PROM), electrically programmable ROM (EPROM), electrically erasable ROM (EEPROM), or flash memory. Volatile memory can include random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to comprise, without being limited to comprising, these and any other suitable types of memory.

Various aspects described herein can be implemented as a method, apparatus, or article of manufacture using standard programming and/or engineering techniques. In addition, various of the aspects disclosed herein also can be implemented by means of program modules or other types of computer program instructions stored in a memory device and executed by a processor, or other combination of hardware and software, or hardware and firmware. Such program modules or computer program instructions can be loaded onto a general purpose computer, a special purpose computer, or another type of programmable data processing apparatus to produce a machine, such that the instructions which execute on the computer or other programmable data processing apparatus create a means for implementing the functionality of disclosed herein.

The terminology “article of manufacture” as used herein is intended to encompass a computer program or other type of machine instructions stored in and accessible from any processor-readable (e.g., computer-readable) device, carrier, or media. For example, processor-readable (e.g., computer readable) media can include magnetic storage devices (e.g., hard drive disk, floppy disk, magnetic strips, or similar), optical discs (e.g., compact disc (CD), digital versatile disc (DVD), blu-ray disc (BD), or similar), smart cards, and flash memory devices (e.g., card, stick, key drive, or similar), and other types of memory devices.

What has been described above includes examples of one or more aspects of the disclosure. It is, of course, not possible to describe every conceivable combination of components or methodologies for purposes of describing these examples, and it can be recognized that many further combinations and permutations of the present aspects are possible. Accordingly, the aspects disclosed and/or claimed herein are intended to embrace all such alterations, modifications and variations that fall within the spirit and scope of the detailed description and the appended claims. Furthermore, to the extent that one or more of the terms “includes,” “including,” “has,” “have,” or “having” is used in either the detailed description or the claims, such term is intended to be inclusive in a manner similar to the term “comprising” as “comprising” is interpreted when employed as a transitional word in a claim.

Numerous other changes, substitutions, variations, alterations, and modifications may be ascertained to one skilled in the art and it is intended that the present disclosure encompass all such changes, substitutions, variations, alterations, and modifications as falling within the scope of the appended claims. In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke 35 U.S.C. § 112(f) as it exists on the date of the filing hereof unless the words “means for” or “steps for” are specifically used in the particular claims; and (b) does not intend, by any statement in the disclosure, to limit this disclosure in any way that is not otherwise reflected in the appended claims.

What is claimed is:

1. A method for performing a hearing test, comprising:
obtaining, by a control system, contextual information associated with a subject and an environment of headphones configured to apply the hearing test, the contextual information including at least one of physiological data of the subject, acoustic characteristics of the environment, or anatomical characteristics of the subject;
causing, by the control system, one or more test signals to be conditioned based on the contextual information, resulting in one or more conditioned test signals;
receiving, by the control system, respective indications of sound pressure level (SPL) of the one or more conditioned test signals;
receiving, by the control system, respective indications of whether the subject heard the one or more conditioned test signals;
generating, by the control system, a hearing profile of the subject based on the respective indications of SPL of the one or more conditioned test signals and the respective indications of whether the subject heard the one or more conditioned test signals; and
retaining, by the control system, the hearing profile and the contextual information in a non-volatile memory device for use in hearing compensation of subsequent sounds emitted by a speaker device of the headphones, with the hearing compensation being based on the hearing profile and the contextual information.

2. The method of claim 1, wherein the obtaining comprises causing detection of an amount of sound leakage passing through the headphones.

3. The method of claim 2, wherein the conditioning comprises compensating for the detected sound leakage by continuously monitoring a feedback microphone response and adjusting noise cancellation filters to maintain a consistent active noise cancellation (ANC) response within a headphone device of the headphones.

4. The method of claim 1, wherein the obtaining comprises causing detection of
a vocalization during the hearing test.

5. The method of claim 4, wherein the method further comprises,
rejecting, by the control system, a test measurement performed during the detected vocalization; and
causing, by the control system, the test measurement to be repeated.

6. The method of claim 1, wherein the obtaining comprises determining tinnitus frequency information corresponding to the subject, with the tinnitus frequency information being indicative of one or more tinnitus frequencies.

7. The method of claim 6, wherein the conditioning comprises at least one of:
adjusting the one or more test signals to avoid having respective frequencies overlapping the one or more tinnitus frequencies, or
increasing an amplitude of the one or more test signals at frequencies within a frequency range that includes the one or more tinnitus frequencies.

8. The method of claim 1, wherein the obtaining comprises causing measurement of at least one physiological attribute of the subject including at least one of heart rate, temperature, or a value of blood oxygen saturation (SpO2).

9. The method of claim 1, wherein the conditioning comprises at least one of:
adjusting at least one of a frequency of the one or more test signals,
adjusting an amplitude of the one or more test signals, or
configuring a spatial rendering of the one or more test signals.

10. The method of claim 9, wherein the configuring the spatial rendering of the one or more test signals comprises pre-filtering the one or more test signals using a head-related transfer function (HRTF) to spatially render the one or more test signals as perceived as originating from one or more directions in three-dimensional space.

11. The method of claim 10, wherein the HRTF corresponds to the subject, the method further comprising,
causing, by the control system, a computing device to capture anatomical characteristics of the subject including at least one of a geometry of a head, geometry of a face, or morphology of ears of the subject; and
causing, by the control system, the computing device, to generate the HRTF based on the captured anatomical characteristics.

12. The method of claim 10, with the one or more directions including multiple directions having respective angles distributed spherically around the subject.

13. A system, comprising:
one or more memory devices;
one or more context modules; and
a control system communicatively coupled with the one or more context modules and the one or more memory devices, the control system configured to:
obtain, using at least one context module of the one or more context modules, contextual information associated with a subject and an environment of headphones configured to apply a hearing test, with the contextual information including at least one of physiological data of the subject, acoustic characteristics of the environment, or anatomical characteristics of the subject;
cause one or more test signals to be conditioned based on the contextual information, resulting in one or more conditioned test signals;
receive respective first indications of SPL of the one or more conditioned test signals;
receive respective second indications of whether the subject heard the one or more conditioned test signals;
generate a hearing profile of the subject based on the respective first indications and the respective second indications; and
retain the hearing profile and the contextual information in a non-volatile memory of the one or more memory devices for use in hearing compensation of subsequent sounds emitted by a speaker device, with the hearing compensation being based on the hearing profile and the contextual information.

14. The system of claim 13, wherein the at least one context module comprises a leakage detection module having an adaptive active noise cancellation (ANC) module, and wherein to obtain the contextual information, the control system is further configured to cause the leakage detection module to detect an amount of sound leakage passing through the headphones.

15. The system of claim 14, wherein the leakage detection module further has a feedback microphone, and wherein conditioning the one or more test signals comprises compensating for detected sound leakage by continuously monitoring a response of the feedback microphone and adjusting noise cancellation filters to maintain a consistent ANC response.

16. The system of claim 13, wherein the at least one context module comprises an own-voice detection module configured to detect a vocalization during the hearing test, wherein to obtain the contextual information, the control system is further configured to cause the own-voice detection module to determine presence or absence of the vocalization during the hearing test.

17. The system of claim 16, wherein the control system is further configured to,
reject a test measurement performed during presence of the vocalization; and
cause the test measurement to be repeated.

18. The system of claim 13, wherein to obtain the contextual information, the control system is further configured to determine tinnitus frequency information corresponding to the subject, with the tinnitus frequency information being indicative of one or more tinnitus frequencies.

19. The system of claim 18, wherein to condition the one or more test signals, the control system is further configured to,
adjust the one or more test signals to avoid having respective frequencies overlapping the one or more tinnitus frequencies, or
increase an amplitude of the one or more test signals at frequencies within a frequency range that includes the one or more tinnitus frequencies.

20. The system of claim 13, wherein the at least one context module comprises a vital sign monitoring (VSM) module configured to measure at least one physiological attribute including at least one of heart rate, temperature, or blood oxygen saturation (SpO2),
wherein to obtain the contextual information, the control system is further configured to cause the VSM module to measure one or more physiological attributes.

21. The system of claim 13, wherein conditioning the one or more test signals comprises configuring a spatial rendering of the one or more test signals, with the configuring including pre-filtering the one or more test signals using a head-related transfer function (HRTF) to spatially render the one or more test signals as perceived as originating from one or more directions in three-dimensional space.
